(19) United States
(12) Patent Application Publication
(10) Pub. No.: US 2026/0348052 A1
(43) Pub. Date: Nov. 5, 2026

(54) Hollow Fibre Membranes and Methods of Manufacture Thereof

(71) Applicant: CELLULAR AGRICULTURE LTD, Llanelli Carmarthenshire (GB)

(72) Inventors: Marianne Jane Ellis, Llanelli Carmarthenshire (GB); Iain Argyle, Llanelli Carmarthenshire (GB); Saeed Mazinani, Llanelli Carmarthenshire (GB)

(21) Appl. No.: 19/478,149

(22) PCT Filed: Apr. 26, 2024
(86) PCT No.: PCT/GB2024/051117

§ 371 Date: Oct. 23, 2025

(30) Foreign Application Priority Data
Apr. 28, 2023 (GB) 2306396.9

(51) Int. Cl. D01D 5/24; C12N 5/00; D01D 5/06; D01D 10/00; D01F 6/22; D01F 6/42; D01F 6/56; D01F 6/76; D04H 1/4282; D04H 1/4326; D04H 1/4391

(52) U.S. Cl. CPC D01D 5/24; C12N 5/0068; D01D 5/06; D01D 10/00; D01F 6/22; D01F 6/42; D01F 6/56; D01F 6/76; D04H 1/4282; D04H 1/4326; D04H 1/43914; D10B 2321/10; D10B 2321/121; D10B 2331/30; D10B 2509/00

(57) ABSTRACT

The invention relates to a method for manufacturing a hollow fibre (e.g. a porous hollow fibre membrane) for tissue engineering using a polymer; a porogen; and a non-toxic solvent. The invention also relates to a composition for forming said hollow fibre, a hollow fibre suitable for use in tissue engineering, and uses of the same. The invention further relates to a method of culturing cells using said hollow fibre.

### FIELD OF THE INVENTION

The present disclosure relates to a method for manufacturing a hollow fibre (e.g. a porous hollow fibre membrane) for tissue engineering. The present disclosure also relates to a composition for forming a hollow fibre for tissue engineering, a hollow fibre suitable for use in tissue engineering, and uses of the same. The present disclosure further relates to a method of culturing cells.

### BACKGROUND

Hollow fibre membranes (HFMs) can be used for different applications in tissue engineering, including cellular agriculture, regenerative medicine and in vitro models, as well as ranging from small-scale operations to large-scale water and wastewater treatment, and gas separation.

Despite the efforts that have been made so far to develop suitable engineering scaffolds (e.g. for production of three-dimensional (3D) tissue constructs) under in vitro conditions, poor transport of nutrients from culture medium to cells remains a major challenge. This challenge could be addressed by using microporous hollow fibre membranes (HFMs) where cells are cultured on the outer surface of fibres and culture medium flows in the lumen of fibres, enabling a uniform distribution of nutrients to cells.

Hollow fibre membranes (HFMs) are typically fabricated by extrusion methods, thus, the solvent system utilised in these methods plays a crucial role in affecting membrane formation and the resulting characteristics. Typical solvents used for this application include N-methyl-2-pyrrolidone (NMP), dimethylacetamide (DMAc) and dimethylformamide (DMF). The use of these solvents is well-established and they are exclusively used in many applications due to their desirable properties, such as their solubility profiles, high polarity, aprotic nature, low viscosity and high miscibility with water. However, there are challenges associated with use of such solvents as they are highly toxic and pose serious risk for human health.

Moreover, toxic solvents, such as NMP, can contribute up to 60% of the total environmental impacts associated with membrane production. It has been reported that approximately 70% of membrane manufactures flush generated waste without purification or dilute wastewater with excess water. Consequently, membrane fabrication is estimated to release over 50 billion litres of contaminated water annually. Utilisation of such toxic solvents creates problems associated with sustainability, waste disposal and environmental burden. Furthermore, there are even more challenges associated with the consequential effects of toxic substances used in the formation of HFMs, limiting potential applications in tissue engineering.

### SUMMARY OF THE INVENTION

According to a first aspect of the present disclosure, there is provided a method for manufacturing a hollow fibre for tissue engineering (e.g. scaffolds). The method comprises a step of extruding a composition for forming a hollow fibre, wherein the composition comprises:
a polymer;
a porogen; and
a non-toxic solvent;
   wherein the non-toxic solvent comprises one or more of dimethyl sulfoxide (DMSO); dimethyl isosorbide (DMI); d-limonene; cyrene (dihydrolevoglucosenone); γ-butyrolactone; isophorone; ethyl lactate; methyl lactate; γ-valerolactone; ethyl acetate; propan-2-ol (IPA); ethanol; methyl 5-(dimethylamino)-2-methyl-5-oxopentanoate; cyclopentanone; and/or N-butyl-2-pyrrolidinone.

Preferably, the hollow fibre is a porous hollow fibre membrane. The non-toxic solvent is preferably a non-cytotoxic solvent and/or a non-CMR solvents (i.e. non-carcinogenic, non-mutagenic, and non-toxic for reproduction).

According to a second aspect of the present disclosure, there is provided a composition for forming a hollow fibre for tissue engineering. The composition comprises:
a polymer;
a porogen; and
a non-toxic solvent;
wherein the non-toxic solvent comprises one or more of dimethyl sulfoxide (DMSO); dimethyl isosorbide (DMI); d-limonene; cyrene (dihydrolevoglucosenone); γ-butyrolactone; isophorone; ethyl lactate; methyl lactate; γ-valerolactone; ethyl acetate; propan-2-ol (IPA); ethanol; methyl 5-(dimethylamino)-2-methyl-5-oxopentanoate; cyclopentanone; and/or N-butyl-2-pyrrolidinone.

Preferably, the composition is used in the method according to the first aspect described herein.

According to a third aspect of the present disclosure, there is provided a method of culturing cells. The method comprises the steps of:
a) providing one or more hollow fibres obtainable from the method according to the first aspect described herein;
b) seeding cells onto the one or more hollow fibres;
c) subjecting the one or more hollow fibres to conditions suitable to allow initial attachment of the cells to an outer surface of each of the one or more hollow fibres;
d) culturing the cells attached to the outer surface of each of the one or more hollow fibres under conditions suitable for proliferation and/or differentiation of the cells.

For the avoidance of doubt, the method of the third aspect of the present disclosure is an in vitro and/or ex vivo method.

### BRIEF DESCRIPTION OF FIGURES

FIGS. 1 (a) and 1 (b): (a) SEM micrographs illustrating the cross section and (b) surface of an exemplary hollow fibre.

FIGS. 2 (a) and 2 (b): graphs illustrating plasma treatment longevity of a flat sheet membrane obtainable from a composition described herein.

FIG. 3: graph illustrating cytocompatibility of flat sheets obtainable from the compositions described herein.

FIG. 4: Images of live-dead cell staining after cell proliferation.

FIGS. 5 (a), 5 (b) and 5 (c): SEM micrographs illustrating the cross section and surface of exemplary hollow fibres.

FIGS. 6 (a), 6 (b), 6 (c) and 6 (d): SEM micrographs illustrating the cross section of exemplary hollow fibres.

FIGS. 7 (a), 7 (b), 7 (c) and 7 (d): SEM micrographs illustrating the cross section of exemplary hollow fibres.

FIG. 8: a graph illustrating fold increase from cell seeding (20% of the inoculum cell number) after 5 days of proliferation on hollow fibres within a hollow fibre bioreactor.

FIG. 9: a graph illustrating the dissolved oxygen profiles which shows the consumption of oxygen represented by the decline in the oxygen concentration in the outlet line as a result of cell growth occurring.

FIG. 10: SEM micrographs illustrating hollow fibres produced using vapour induced phase separation with parameters set out in Table 4 (a) to (b).

FIG. 11: Schematic diagram of vapour induced phase separation set up.

### DETAILED DESCRIPTION

According to a first aspect of the present disclosure, there is provided a method for manufacturing a hollow fibre for tissue engineering. In some examples, the hollow fibres of the invention may be referred to as tissue engineering or cell culture fibres. That is to say that the hollow fibres described herein have specific properties that optimise their use as structures for culturing both adherent and non-adherent cells. This is in contrast to hollow fibres and membranes used for filtration, in particular nanofiltration purposes. The membranes used in the fibres described herein for cell culturing are configured to allow rapid diffusion of nutrients and waste products to and from the cells (e.g. are porous). The hollow fibres described herein may have pore sizes small enough to retain cells within a bioreactor. However, for example mammalian cells, as used in cell culturing methods are relatively large, typically between 7 μm and 100 μm. Hence, provided that the pores are smaller than the cells being cultured (e.g. less than 7 μm or less than 100 μm, depending on cells being cultured), hollow fibres optimised for cell culturing can have pore sizes and pore size distributions that are significantly larger than those which are optimised for filtration of very small species, such as proteins. The relatively larger pore sizes, as compared to the pores in typical membranes optimised for filtration (e.g. nanofiltration), retain the cultured cells whilst allowing a more rapid and easy diffusion of nutrients and waste products. As such, in some examples, the hollow fibres are not suitable for filtration or are not for filtration. In some examples, there is provided a method for manufacturing a tissue engineering hollow fibre. In some examples, there is provided a method for manufacturing a cell culture hollow fibre. In some examples, the hollow fibres are configured for cell culture and/or tissue engineering. In some examples, the hollow fibres comprise an average pore diameter of less than 100 μm. In some examples, the hollow fibres comprise an average pore diameter of less than 50 μm. In some examples, the hollow fibres comprise an average pore diameter of less than 20 μm. In some examples, the hollow fibres comprise an average pore diameter of less than 10 μm. In some examples, the hollow fibres comprise an average pore diameter of less than 7 μm.

The method for manufacturing a hollow fibre (e.g. a porous hollow fibre membrane) according to the present disclosure provides an improved method having excellent applicability to applications for tissue engineering (e.g. scaffolds), whilst mitigating the use of toxic solvents (e.g. cytotoxic solvents, CMR solvents, solvents that pose serious risk for human health). Such hollow fibres, obtainable according to the present disclosure, exhibit good properties for use in tissue engineering. At the very least these properties are similar to, but are often better than, HFMs obtainable from known methods using toxic solvents.

Suitable applications in tissue engineering include cell proliferation, cell differentiation and/or culturing or harvesting of cells.

The method comprises a step of extruding a composition for forming a hollow fibre, wherein the composition comprises, or consists essentially of:
a polymer;
a porogen; and
a non-toxic solvent.

In principle, the step of extruding the composition can be achieved by any suitable fabrication technique (e.g. wet extrusion technique, dry-wet spinning technique, thermally induced phase separation technique) for forming a hollow fibre, particularly a hollow fibre membrane. In some examples, the step of extruding the composition is achieved by a dry-wet spinning technique.

The step of extruding a composition for forming a hollow fibre is to produce a hollow fibre extrudate, preferably a hollow fibre membrane extrudate. The hollow fibre extrudate is in the form of a hollow fibre, particularly a hollow fibre membrane. As the hollow fibre extrudate is an extruded form of the composition for forming the hollow fibre, then the extrudate still comprises the non-toxic solvent.

In general, the step of extruding the composition for forming a hollow fibre is a step of extruding the composition with or through a spinneret to form the hollow fibre. In other words, the step of extruding a composition for forming a hollow fibre is a step of spinning the composition with a spinneret to form the hollow fibre.

As the composition of the invention comprises a solvent (e.g. the non-toxic solvent), the hollow fibre is typically formed by a wet spinning technique (e.g. a dry-wet spinning technique). Wet-spinning techniques for forming polymer fibres, particularly hollow fibre membranes, are known in the art. The non-toxic solvent in the composition for forming the hollow fibre dissolves the polymer and is sometimes referred to as a “dope” or a “spinning solvent”.

The method of the first aspect of the invention may further comprise the step of submerging the hollow fibre extrudate in a nonsolvent. The hollow fibre extrudate, such as a hollow fibre membrane extrudate, is submerged in the nonsolvent to produce a hollow fibre, particularly a hollow fibre membrane.

The purpose of submerging the hollow fibre extrudate in the nonsolvent is to bring about an exchange between the non-toxic solvent and the nonsolvent that causes a phase inversion, which leads to solvent removal, solidification and precipitation of a hollow fibre, preferably a hollow fibre membrane.

Thus, the method may include extruding a composition for forming a hollow fibre into a coagulation bath comprising, or consisting essentially of, a nonsolvent (e.g. to submerge the hollow fiber extrudate in the nonsolvent). In some examples, the nonsolvent comprises water.

Prior to the step of extruding a composition for forming a hollow fibre into a coagulation bath, the method may comprise the step of exposing the composition to a vapor (e.g. to induce phase separation by Vapour-induced Phase Separation (VIPS)), wherein the vapour phase comprises a nonsolvent. The nonsolvent in the vapor phase may comprise, or consist essentially of, the same or different nonsolvent in the coagulation bath. In some examples, the vapour may comprise a solvent. In some examples, the vapour comprises a non-toxic solvent as described herein. In some examples, the vapour comprises a mixture of a non-solvent as described herein and a non-toxic solvent as described herein.

In short, VIPS may include passing the composition (dope solution) and a bore liquid through a coaxial nozzle (e.g. spinneret). The composition is then extruded into a tunnel. The tunnel comprises a vapour (e.g. non-solvent vapour such as water vapour) therein which contacts the extruded composition. The vapour may flow in to the tunnel via an entry port and exit the tunnel via an exit port. The temperature of the vapour may be controlled (e.g. by heating or cooling apparatus). The entry port may be at a bottom of the tunnel and exit port at the top of the tunnel (i.e. the entry and exit are located at opposing ends of the tunnel). The flow of the vapour may be a counter-current to the flow of the extradited composition or may be a co-current flow with the flow of the extradited composition. The rate of extrusion and the length of the air gap define the residence time of exposure to the non-solvent vapour e.g. humid air leads to a slowing of the non-solvent from the vapour phase into the extruded dope solution. Modifying the exposure time to the vapour before the fibre enters the coagulation bath enables control of the porous structure. The air gap is characterised by the distance from the nozzle tip (spinneret tip) to the coagulation bath. For example, includes the length of the tunnel and may also include any distance between the end of the tunnel and the coagulation bath. The air gap may extend beyond the length of the tunnel by positioning the spinneret height and tunnel height above the bath as necessary to create the pore structure required. Other factors such as the relative humidity, air temperature and vapour velocity can also be used to modify the final properties of the hollow fibres produce (for example of a setup see FIG. 11).

In some examples, the method uses a bore fluid comprising a solvent, such as a non-toxic solvent as described herein. In some examples, the bore fluid comprises a non-solvent and a non-toxic solvent. In some examples, the bore fluid may include from about 10% to 90% non-toxic solvent. For example, the bore liquid may comprise from a ratio of non-toxic solvent to non-solvent of 10% to 90%. For example, the bore liquid may comprise from a ratio of non-toxic solvent to non-solvent of 10% to 75%. As such, the fluid may comprise from about 10% to 90% non-solvent. As such, the fluid may comprise from about 25% to 90% non-solvent. In some examples the non-solvent is water. For example, the bore fluid may comprise from about 10% to 75% non-toxic solvent in water. In some examples, the bore fluid may comprise 25% non-toxic solvent and 75% non-solvent. For example, 25% non-toxic solvent and 75% water. For example, 25% N-butyl-2-pyrrolidinone (e.g. TamiSolve™ NxG and 75% water. Bore fluid or liquid refers to an internal fluid (sometimes referred to as coagulant) pumped through the inner tube of a spinneret used for the fabrication of hollow fibres by the dry/wet spinning technique or the wet spinning technique.

In some examples, the method includes VIPS wherein the bore fluid comprises a non-toxic solvent as described herein and a non-solvent. In some examples, the bore fluid may include from about 10% to 90% non-toxic solvent. In some examples, the bore fluid may include from about 10% to 75% non-toxic solvent. In some examples, the bore fluid may comprise 25% non-toxic solvent and 75% non-solvent. For example, 25% non-toxic solvent and 75% water. For example, 25% N-butyl-2-pyrrolidinone (e.g. TamiSolve™ NxG and 75% water).

In some examples, the method may include the use of VIPS wherein the air gap between the spinneret and coagulation bath is from about 0.5 cm to about 100 cm. In some examples, the air gap is from about 4 cm to about 70 cm. In some examples, the air gap is from about 50 cm to about 70 cm. In some examples, the air gap is about 65 cm.

In some examples, the residence time of the extrudate to the vapour (i.e. time the extrudate is in the tunnel and the space between the end of the tunnel and the coagulation bath) may be at least 0.5 seconds. For example, residence time in the air gap may be from about 0.5 s to about 3.5 s. Preferably, residence time in the air gap may be about 1 s to about 3 s. The residence time in the air gap may be altered or changed by changing the length of the air gap and/or by changing the velocity at which the extrudate travels through the air gap.

In some examples, the air gap may comprise a solvent or non-solvent. For example, the air gap and/or the tunnel may comprise a non-toxic solvent as described herein. For example, the air gap and/or the tunnel may comprise a non-solvent as described herein. In some examples, the relative humidity (or relative % saturation of the air with non-toxic solvent as described herein or non-solvent as described herein) in the air gap and/or the tunnel when using VIPS may be greater than 70% relative humidity. For example, 70, 75, 80, 85, 90, 95 or 100% relative humidity. For example, the air gap and/or the tunnel may have a relative humidity from about 70% to about 100%. For example, the air gap and/or the tunnel may have at least 70% relative saturation of the air with non-toxic solvent as described herein. For example, the air gap and/or the tunnel may have from about 70% to about 100% relative saturation of the air with non-toxic solvent as described herein. The relative humidity or % saturation in the air gap and/or the tunnel may be measured using any suitable method. For example, using a Rotronic HC2A-S probe available from PST.

The nonsolvent is for solidifying and/or precipitating the hollow fibre extrudate, preferably the hollow fibre membrane extrudate. The nonsolvent may also dissolve or disperse, preferably dissolve, the porogen. The method may further comprise a step of removing the hollow fiber, preferably the hollow fiber membrane, from the nonsolvent (e.g. the coagulation bath comprising, or consisting essentially of, the nonsolvent) and optionally drying the hollow fiber, preferably the hollow fiber membrane.

The nonsolvent that is used will depend on the non-toxic solvent and the polymer in the composition for forming the hollow fiber. In some examples, the non-solvent is water. For example, if the non-toxic solvent comprises acetone, a suitable nonsolvent may be water e.g. reverse osmosis (R.O.) water.

Generally, the polymer is soluble in the non-toxic solvent. The polymer is soluble in the non-toxic solvent at room temperature (e.g. about 20.0 °C). Additionally or alternatively, it is preferred that the polymer is soluble in the non-toxic solvent at the conditions at which the extrusion step of the method is performed.

The acronym “CMR” as used herein refers to carcinogenic, mutagenic or reprotoxic. The term “CMR” is used in legislation, for example from the European Commission, which govern the limits of certain substances in products.

Typically, the non-toxic solvent is a non-cytotoxic solvent. As used herein, the term “non-cytotoxic” refers to a substance, particularly a solvent, that is not toxic to living cells. For example, a non-cytotoxic substance can refer to any substance relating to or producing a toxic effect to living cells (e.g. eukaryotic cells). Toxic effects include, but are not limited to, apoptosis and necrosis. Preferably, the non-toxic solvent does not exhibit cytotoxic effects to living cells (e.g. eukaryotic cells).

The non-toxic solvent may be a non-reprotoxic solvent. As used herein, the term “non-reprotoxic” means a substance, particularly a solvent, that does not cause reproductive toxicity.

The non-toxic solvent may be a non-CMR solvent. The term “non-CMR solvent” refers to a solvent that is not carcinogenic, not mutagenic and not reprotoxic (i.e. toxic for reproduction).

The non-toxic solvent may have one or more of the following properties: biodegradable, eco-friendly, sustainable, having good or excellent toxicological and/or eco-toxicological profiles. Preferably, the non-toxic solvent is not hazardous (e.g. including being harmful in contact with skin, harmful if inhaled, harmful if swallowed, cause serious eye irritation, cause severe skin burns and eye damage, may damage fertility or the unborn child). The non-toxic solvent is preferably not toxic in humans and animals.

It is preferred that the non-toxic solvent is a non-cytotoxic solvent and/or a non-CMR solvent, preferably a non-cytotoxic solvent and a non-CMR solvent. Preferably, the non-toxic solvent is not teratogenic.

The non-toxic solvent may be a non-toxic bio-based solvent (also referred herein as a green solvent). As used herein, the term “bio-solvent” or “green solvent” refers to a solvent with one or more of the following properties: it is not, or does not produce, volatile organic compounds (VOCs), is obtainable from a renewable source, such as biomass, is bio-based, biodegradable and recyclable.

The non-toxic solvent may comprise, or consist essentially of, a mixture of solvents (e.g. two or more different solvents). When the non-toxic solvent comprises, or consists essentially of, a mixture of solvents, it is to be understood that the terms “non-toxic”, “non-cytotoxic”, “non-reprotoxic” and “non-CMR” when used to define the solvent refer to the overall solvent mixture.

The non-toxic solvent may be selected from or comprise one or more of acetone; dimethyl sulfoxide (DMSO); dimethyl isosorbide (DMI); d-limonene; Cyrene™ (dihydrolevoglucosenone); methyl oleate; triethyl phosphate (TEP); γ-butyrolactone; 1,2-butylene carbonate; methyl ethyl ketone (MEK); palm oil; cyclopentanone; dioctyl adipate; isophorone; acetyl triethyl citrate (ATEC); oleic acid; ethyl lactate; methyl lactate; acetyl tributyl citrate; triethylcitrate; triethylene glycol diacetate; triethyleneglycol diacetate acetone; γ-valerolactone; ethyl acetate; propan-2-ol (IPA); ethanol; methyl 5-(dimethylamino)-2-methyl-5-oxopentanoate (e.g. Rhodiasolv™ Polarclean (CAS-No. 174627-68-9)); N-butyl-2-pyrrolidinone (e.g. TamiSolve™ NxG); and a mixture of two or more thereof.

It is preferred that the non-toxic solvent is acetone; dimethyl sulfoxide (DMSO); dimethyl isosorbide (DMI); d-limonene; methyl 5-(dimethylamino)-2-methyl-5-oxopentanoate (e.g. Rhodiasolv™ Polarclean (CAS-No. 174627-68-9)); N-butyl-2-pyrrolidinone (e.g. TamiSolve™ NxG); or a mixture of two or more thereof. More preferably, the non-toxic solvent is acetone; dimethyl isosorbide (DMI); methyl 5-(dimethylamino)-2-methyl-5-oxopentanoate (e.g. Rhodiasolv™ Polarclean (CAS-No. 174627-68-9)); N-butyl-2-pyrrolidinone (e.g. TamiSolve™ NxG); or a mixture of two or more thereof.

The non-toxic solvent may comprise, or consist essentially of, a single solvent, such as a solvent defined hereinabove. It is preferred that the single solvent is dimethyl isosorbide (DMI); methyl 5-(dimethylamino)-2-methyl-5-oxopentanoate (e.g. Rhodiasolv™ Polarclean (CAS-No. 174627-68-9)); N-butyl-2-pyrrolidinone (e.g. TamiSolve™ NxG), more preferably methyl 5-(dimethylamino)-2-methyl-5-oxopentanoate (e.g. Rhodiasolv™ Polarclean (CAS-No. 174627-68-9)) or N-butyl-2-pyrrolidinone (e.g. TamiSolve™ NxG), and even more preferably N-butyl-2-pyrrolidinone (e.g. TamiSolve™ NxG).

When the non-toxic solvent comprises, or consists essentially of, a mixture of solvents, it is preferred that the non-toxic solvent comprises, or consists essentially of, two solvents (e.g. a mixture of two different solvents), such as a first solvent and a second solvent.

For the avoidance of doubt, the non-toxic solvent does not comprise a toxic solvent (e.g. a CMR solvent, a cytotoxic solvent and/or a reprotoxic solvent). Thus, the non-toxic solvent does not comprise N-methyl-2-pyrrolidone (NMP), dimethylacetamide (DMAc) or dimethylformamide (DMF). More preferably, the non-toxic solvent does not comprise N-methyl-2-pyrrolidone (NMP). In some examples, the non-toxic solvent is less cytotoxic than NMP, DMAc and/or DMF to non-tumorigenic and malignant cells, bacteria and fungi. Preferably, the non-toxic solvent is less cytotoxic than DMF to non-tumorigenic and malignant cells, bacteria and fungi. Various toxicological studies have been performed on solvents in the art, such as NMP, to measure their toxicity. Various toxicological markers may include mutagenic effects on microorganisms or reproductive, developmental or tumorigenic effects on experimental animals In some examples, the non-toxic solvent is less cytotoxic than DMF to endothelial cells (e.g. human umbilical vein endothelial cells). In some examples, the non-toxic solvent does not comprise a toxic dipolar aprotic solvent and/or a teratogenic compound.

A suitable non-toxic solvent can be selected depending on the polymer in the composition for forming the hollow fiber. Typically, suitability of the non-toxic solvent for dissolving a polymer can be measured by calculating the Relative Energy Difference (RED) value, using The Hansen Solubility Parameter (HSP), according to the following formulas:

Rₐ²=4(δ_{d2}−δₐ₁)²+(δₚ₂−δₚ₁)²+(δₕ₂−δₕ₁)²

RED=Rₐ/R₀
wherein:
δₐ is dispersion forces
δₚ is polar interaction
δₕ is hydrogen bonding
Rₐ is HSP distance
R₀ is the solubility radius (interaction radius) of polymer
RED Relative energy distance
Subscript 2 corresponds to the non-toxic solvent
Subscript 1 corresponds to the polymer

In the case that a mixture of solvents (e.g. two solvents or solvent blend) is used as the non-toxic solvent, the RED is calculated using the total sum of the mixture of solvents. The RED value may be used to select a suitable non-toxic solvent to dissolve a selected polymer. It is preferred that the non-toxic solvent and polymer RED value is less than 1.0.

The non-toxic solvent is preferably aprotic. The non-toxic solvent may be a polar aprotic solvent. The non-toxic solvent may comprise a compound having an oxygen-containing functional group, such as a carbonyl group.

When the non-toxic solvent comprises, or consists essentially of, two solvents (e.g. two different solvents), then a first solvent and a second solvent may each independently be selected from acetone; dimethyl sulfoxide (DMSO); dimethyl isosorbide (DMI); d-limonene; cyrene (dihydrolevoglucosenone); methyl oleate; triethyl phosphate (TEP); γ-butyrolactone; 1,2-butylene carbonate; methyl ethyl ketone (MEK); ‘palm oil’; cyclopentanone; dioctyl adipate; isophorone; acetyl triethyl citrate (ATEC); oleic acid; ethyl lactate; methyl lactate; acetyl tributyl citrate; triethylcitrate; triethylene glycol diacetate; triethyleneglycol diacetate acetone; γ-valerolactone; ethyl acetate; propan-2-ol (IPA); ethanol; methyl 5-(dimethylamino)-2-methyl-5-oxopentanoate (e.g. Rhodiasolv™ Polarclean (CAS-No. 174627-68-9)) and N-butyl-2-pyrrolidinone (e.g. TamiSolve™ NxG). It is preferred that the first solvent and the second solvent is each independently selected from acetone; dimethyl sulfoxide (DMSO); dimethyl isosorbide (DMI); d-limonene; cyrene; N-butyl-2-pyrrolidinone; methyl 5-(dimethylamino)-2-methyl-5-oxopentanoate; and a mixture of two or more thereof. More preferably, the first solvent and the second solvent is each independently selected from acetone; dimethyl isosorbide (DMI); d-limonene; cyrene; N-butyl-2-pyrrolidinone; methyl 5-(dimethylamino)-2-methyl-5-oxopentanoate; and a mixture of two or more thereof.

In some examples, the solvent comprises one or more of dimethyl sulfoxide (DMSO); dimethyl isosorbide (DMI); d-limonene; cyrene (dihydrolevoglucosenone); γ-butyrolactone; isophorone; ethyl lactate; methyl lactate; γ-valerolactone; ethyl acetate; propan-2-ol (IPA); ethanol; methyl 5-(dimethylamino)-2-methyl-5-oxopentanoate; cyclopentanone; and/or N-butyl-2-pyrrolidinone.

In some examples, the solvent comprises or consists of dimethyl sulfoxide (DMSO).

In some examples, the solvent comprises or consists of dimethyl isosorbide (DMI). In some examples, the solvent comprises or consists of d-limonene.

In some examples, the solvent comprises or consists of cyrene (dihydrolevoglucosenone).

In some examples, the solvent comprises or consists of γ-butyrolactone.

In some examples, the solvent comprises or consists of isophorone.

In some examples, the solvent comprises or consists of isophorone.

In some examples, the solvent comprises or consists of ethyl lactate.

In some examples, the solvent comprises or consists of methyl lactate.

In some examples, the solvent comprises or consists of γ-valerolactone.

In some examples, the solvent comprises or consists of ethyl acetate.

In some examples, the solvent comprises or consists of propan-2-ol (IPA).

In some examples, the solvent comprises or consists of ethanol.

In some examples, the solvent comprises or consists of methyl 5-(dimethylamino)-2-methyl-5-oxopentanoate.

In some examples, the solvent comprises or consists of N-butyl-2-pyrrolidinone.

In some examples, the solvent comprises or consists of cyclopentanone.

When the non-toxic solvent comprises, or consists essentially of, two solvents, then the non-toxic solvent may include the first solvent and the second solvent in a wt. % ratio of about greater than 0 but less than 100. For example, 1:99, 5:95 to about 95:5 (e.g. about 10:90 to about 90:10), more preferably about 20:80 to about 80:20, and even more preferably about 25:75 to about 75:25. The non-toxic solvent may include the first solvent and the second solvent in a wt. % ratio of 30:70, 35:65, 40:60, 45:55, 50:50, 55:45, 60:40, 65:35, 70:30. More preferably, the wt. % ratio of first solvent and the second solvent is about 25:75, 50:50 or 75:25.

In one example, the non-toxic solvent comprises methyl 5-(dimethylamino)-2-methyl-5-oxopentanoate. The non-toxic solvent can comprise methyl 5-(dimethylamino)-2-methyl-5-oxopentanoate in an amount from about 25 wt. % to about 100 wt. %, e.g. from about 50 wt. % to about 75 wt. %. In some examples, the non-toxic solvent comprises methyl 5-(dimethylamino)-2-methyl-5-oxopentanoate in an amount of 95 wt. % to about 100 wt. %. For example, the non-toxic solvent comprises methyl 5-(dimethylamino)-2-methyl-5-oxopentanoate in an amount of about 20 wt. %, 25 wt. %, 30 wt. %, 35 wt. % 40 wt. %, 45 wt. %, 50 wt. %, 55 wt. %, 60 wt. % 65 wt. %, 70 wt. %, 75 wt. %, 80 wt. %, 85 wt. % 90 wt. %, 95 wt. % or 100 wt. %.

In some examples, the non-toxic solvent can comprise methyl 5-(dimethylamino)-2-methyl-5-oxopentanoate (e.g. (e.g. Rhodiasolv® Polarclean (CAS-No. 174627-68-9)) as a first solvent and further comprises a second solvent. The second solvent can be selected from the group consisting of acetone; dimethyl sulfoxide (DMSO); dimethyl isosorbide (DMI); (+)-limonene; cyrene; methyl oleate; triethyl phosphate (TEP); γ-butryolactone; 1,2-butylene carbonate; methyl ethyl ketone (MEK); palm oil; cyclopentanone; dioctyl adipate; isophorone; acetyl triethyl citrate (ATEC); oleic acid; ethyl lactate; methyl lactate, dimethylsulfoxide, γ-butyrolactone; acetyl citrate; triethylcitrate; triethylene glycol diacetate; triethyleneglycol diacetate acetone; γ-valerolactone; ethyl acetate; propan-2-ol (IPA), ethanol and N-butyl-2-pyrrolidinone (e.g. TamiSolve® NxG), or mixtures thereof. Preferably, the second solvent is selected from acetone; dimethyl sulfoxide (DMSO); dimethyl isosorbide (DMI); d-limonene; and N-butyl-2-pyrrolidinone (e.g. TamiSolve™ NxG).

The composition can comprise methyl 5-(dimethylamino)-2-methyl-5-oxopentanoate and a second solvent in a wt. % ratio of about greater than 0 but less than 100. For example, 1:99, 5:95 to about 95:5 (e.g. about 10:90 to about 90:10), more preferably about 20:80 to about 80:20, and even more preferably about 25:75 to about 75:25. The non-toxic solvent may include methyl 5-(dimethylamino)-2-methyl-5-oxopentanoate and the second solvent in a wt. % ratio of 30:70, 35:65, 40:60, 45:55, 50:50, 55:45, 60:40, 65:35, 70:30. More preferably, the wt. % ratio of methyl 5-(dimethylamino)-2-methyl-5-oxopentanoate and the second solvent is about 25:75, 50:50 or 75:25. In other examples, the non-toxic solvent is methyl 5-(dimethylamino)-2-methyl-5-oxopentanoate. Preferably, the non-toxic solvent is Rhodiasolv® Polarclean (CAS-No. 174627-68-9).

In some examples, the non-toxic solvent comprises N-alkyl-2-pyrrolidone, wherein the alkyl group is selected from C2-C12, preferably C2-C8, more preferably C3-C8, even more preferably, C4-C8. The alkyl group may be linear or branched, saturated or unsaturated. Preferably, the alkyl group is linear. For example, the alkyl group can be linear and selected from C4-C8. More preferably, the alkyl group is linear and selected from C4-C6. Even more preferably, the non-toxic solvent comprises N-butyl-2-pyrrolidinone. The non-toxic solvent can comprise N-alkyl-2-pyrrolidone (e.g. N-butyl-2-pyrrolidinone) in an amount from about 25 wt. % to about 100 wt. %, e.g. from about 50 wt. % to about 75 wt. %. In some examples, the non-toxic solvent comprises N-alkyl-2-pyrrolidone (e.g. N-butyl-2-pyrrolidinone) in an amount of 95 wt. % to about 100 wt. %. For example, the non-toxic solvent comprises N-alkyl-2-pyrrolidone (e.g. N-butyl-2-pyrrolidinone) in an amount of about 20 wt. %, 25 wt. %, 30 wt. %, 35 wt. % 40 wt. %, 45 wt. %, 50 wt. %, 55 wt. %, 60 wt. % 65 wt. %, 70 wt. %, 75 wt. %, 80 wt. %, 85 wt. % 90 wt. %, 95 wt. % or 100 wt. %.

In some examples, the non-toxic solvent can comprise N-alkyl-2-pyrrolidone (e.g. N-butyl-2-pyrrolidinone) and a second solvent. The second solvent can be selected from the group consisting of acetone; dimethyl sulfoxide (DMSO); dimethyl isosorbide (DMI); (+)-limonene; cyrene; methyl oleate; triethyl phosphate (TEP); γ-butryolactone; 1,2-butylene carbonate; methyl ethyl ketone (MEK); ‘palm oil’; cyclopentanone; dioctyl adipate; isophorone; acetyl triethyl citrate (ATEC); oleic acid; ethyl lactate; methyl lactate, dimethylsulfoxide, γ-butyrolactone; acetyl tributyl citrate; triethylcitrate; triethylene glycol diacetate; triethyleneglycol diacetate acetone; γ-valerolactone; ethyl acetate; propan-2-ol (IPA), ethanol and methyl 5-(dimethylamino)-2-methyl-5-oxopentanoate (e.g. Rhodiasolv® Polarclean (CAS-No. 174627-68-9)), or mixtures thereof. Preferably, the second solvent is selected from acetone; dimethyl sulfoxide (DMSO); dimethyl isosorbide (DMI); d-limonene and methyl 5-(dimethylamino)-2-methyl-5-oxopentanoate (e.g. Rhodiasolv™ Polarclean (CAS-No. 174627-68-9)).

The composition can comprise N-alkyl-2-pyrrolidone (e.g. N-butyl-2-pyrrolidinone) and a second solvent in a wt. % ratio of about 5:95 to about 95:5 (e.g. about 10:90 to about 90:10), more preferably about 20:80 to about 80:20, and even more preferably about 25:75 to about 75:25. The non-toxic solvent may include N-alkyl-2-pyrrolidone (e.g. N-butyl-2-pyrrolidinone) and the second solvent in a wt. % ratio of 30:70, 35:65, 40:60, 45:55, 50:50, 55:45, 60:40, 65:35, 70:30. More preferably, the wt. % ratio of N-alkyl-2-pyrrolidone (e.g. N-butyl-2-pyrrolidinone) and the second solvent is about 25:75, 50:50 or 75:25. In other examples, the non-toxic solvent is N-butyl-2-pyrrolidinone. Preferably, the non-toxic solvent is (consists essentially of) TamiSolve® NxG.

In some examples, the non-toxic solvent comprises dimethyl isosorbide. The non-toxic solvent can comprise dimethyl isosorbide in an amount from about 25 wt. % to about 100 wt. %, e.g. from about 50 wt. % to about 75 wt. %. In some examples, the solvent comprises dimethyl isosorbide in an amount of 95 wt. % to about 100 wt. %. For example, the non-toxic solvent comprises dimethyl isosorbide in an amount of about 20 wt. %, 25 wt. %, 30 wt. %, 35 wt. % 40 wt. %, 45 wt. %, 50 wt. %, 55 wt. %, 60 wt. % 65 wt. %, 70 wt. %, 75 wt. %, 80 wt. %, 85 wt. % 90 wt. %, 95 wt. % or 100 wt. %.

In some examples, the non-toxic solvent can comprise dimethyl isosorbide as the first solvent, and further comprise a second solvent. The second solvent can be selected from the group consisting of acetone; dimethyl sulfoxide (DMSO); (+)-limonene; cyrene; methyl oleate; triethyl phosphate (TEP); γ-butryolactone; 1,2-butylene carbonate; methyl ethyl ketone (MEK); ‘palm oil’; cyclopentanone; dioctyl adipate; isophorone; acetyl triethyl citrate (ATEC); oleic acid; ethyl lactate; methyl lactate, dimethylsulfoxide, γ-butyrolactone; acetyl tributyl citrate; triethylcitrate; triethylene glycol diacetate; triethyleneglycol diacetate acetone; γ-valerolactone; ethyl acetate; propan-2-ol (IPA), ethanol, methyl 5-(dimethylamino)-2-methyl-5-oxopentanoate (e.g. Rhodiasolv® Polarclean (CAS-No. 174627-68-9)) and N-butyl-2-pyrrolidinone (e.g. TamiSolve® NxG), or mixtures thereof. Preferably, the second solvent is selected from acetone; dimethyl sulfoxide (DMSO); d-limonene; methyl 5-(dimethylamino)-2-methyl-5-oxopentanoate (e.g. Rhodiasolv™ Polarclean (CAS-No. 174627-68-9)) and N-butyl-2-pyrrolidinone (e.g. TamiSolve™ NxG).

The composition can comprise dimethyl isosorbide and a second solvent in a wt. % ratio of about greater than 0 but less than 100. For example, 1:99, 5:95 to about 95:5 (e.g. about 10:90 to about 90:10), more preferably about 20:80 to about 80:20, and even more preferably about 25:75 to about 75:25. The non-toxic solvent may include dimethyl isosorbide and the second solvent in a wt. % ratio of 30:70, 35:65, 40:60, 45:55, 50:50, 55:45, 60:40, 65:35, 70:30. More preferably, the wt. % ratio of dimethyl isosorbide and the second solvent is about 25:75, 50:50 or 75:25. In other examples, the non-toxic solvent is dimethyl isosorbide. Preferably, the non-toxic solvent is (consists essentially of) dimethyl isosorbide.

In some examples, the non-toxic solvent comprises acetone. The non-toxic solvent can comprise acetone in an amount from about 25 wt. % to about 100 wt. %, e.g. from about 50 wt. % to about 75 wt. %. In some examples, the non-toxic solvent comprises acetone in an amount of 95 wt. % to about 100 wt. %. For example, the non-toxic solvent comprises acetone in an amount of about 20 wt. %, 25 wt. %, 30 wt. %, 35 wt. % 40 wt. %, 45 wt. %, 50 wt. %, 55 wt. %, 60 wt. % 65 wt. %, 70 wt. %, 75 wt. %, 80 wt. %, 85 wt. % 90 wt. %, 95 wt. % or 100 wt. %. In some examples, the non-toxic solvent is acetone.

In other examples, the non-toxic solvent comprises acetone as a first solvent and further comprises a second solvent. The second solvent can be selected from dimethyl sulfoxide (DMSO); dimethyl isosorbide (DMI); d-limonene; cyrene (dihydrolevoglucosenone); methyl oleate; triethyl phosphate (TEP); γ-butyrolactone; 1,2-butylene carbonate; methyl ethyl ketone (MEK); palm oil; cyclopentanone; dioctyl adipate; isophorone; acetyl triethyl citrate (ATEC); oleic acid; ethyl lactate; methyl lactate; acetyl tributyl citrate; triethyl citrate; triethylene glycol diacetate; triethyleneglycol diacetate acetone; γ-valerolactone; ethyl acetate; propan-2-ol (IPA); ethanol; methyl 5-(dimethylamino)-2-methyl-5-oxopentanoate (e.g. Rhodiasolv™ Polarclean (CAS-No. 174627-68-9)); N-butyl-2-pyrrolidinone (e.g. TamiSolve™ NxG); and a mixture of two or more thereof. Preferably, the second solvent is selected from dimethyl sulfoxide (DMSO); dimethyl isosorbide (DMI); d-limonene; methyl 5-(dimethylamino)-2-methyl-5-oxopentanoate (e.g. Rhodiasolv™ Polarclean (CAS-No. 174627-68-9)) and N-butyl-2-pyrrolidinone (e.g. TamiSolve™ NxG). More preferably, the second solvent is selected from dimethyl isosorbide (DMI); methyl 5-(dimethylamino)-2-methyl-5-oxopentanoate (e.g. Rhodiasolv™ Polarclean (CAS-No. 174627-68-9)) and N-butyl-2-pyrrolidinone (e.g. TamiSolve™ NxG).

The non-toxic solvent can be selected from acetone, N-butyl-2-pyrrolidinone, dimethyl isosorbide (DMI) or mixtures thereof. In some examples, the non-toxic solvent comprises acetone and N-alkyl-2-pyrrolidone (e.g. N-butyl-2-pyrrolidinone), preferably in an amount of about greater than 0 but less than 100. For example, 1:99, 5:95 to about 95:5 (e.g. about 10:90 to about 90:10), more preferably about 20:80 to about 80:20, and even more preferably about 25:75 to about 75:25. For example, the non-toxic solvent may comprise acetone and N-alkyl-2-pyrrolidone (e.g. N-butyl-2-pyrrolidinone) in a wt. % ratio of 30:70, 35:65, 40:60, 45:55, 50:50, 55:45, 60:40, 65:35, 70:30. Preferably, the wt. % ratio of acetone to N-alkyl-2-pyrrolidone (e.g. N-butyl-2-pyrrolidinone) is about 25:75, 50:50 or 75:25.

In other examples, the non-toxic solvent comprises acetone and dimethyl isosorbide (DMI). For example, the non-toxic solvent may comprise acetone and DMI in an amount of about 5:95 to about 95:5 (e.g. about 10:90 to about 90:10), more preferably about 20:80 to about 80:20, and even more preferably about 25:75 to about 75:25. For example, the non-toxic solvent may comprise acetone and DMI in a wt. % ratio of 30:70, 35:65, 40:60, 45:55, 50:50, 55:45, 60:40, 65:35, 70:30. Preferably, the wt. % ratio of acetone to DMI is about 25:75, 50:50 or 75:25.

In other examples, the non-toxic solvent comprises acetone and methyl 5-(dimethylamino)-2-methyl-5-oxopentanoate. For example, the non-toxic solvent may comprise acetone and methyl 5-(dimethylamino)-2-methyl-5-oxopentanoate in an amount of about 5:95 to about 95:5 (e.g. about 10:90 to about 90:10), more preferably about 20:80 to about 80:20, and even more preferably about 25:75 to about 75:25. For example, the non-toxic solvent may comprise acetone and methyl 5-(dimethylamino)-2-methyl-5-oxopentanoate in a wt. % ratio of 30:70, 35:65, 40:60, 45:55, 50:50, 55:45, 60:40, 65:35, 70:30. Preferably, the wt. % ratio of acetone to methyl 5-(dimethylamino)-2-methyl-5-oxopentanoate is about 25:75, 50:50 or 75:25.

In some examples, the non-toxic solvent comprises a first solvent and a second solvent, wherein the first solvent is selected from dimethyl isosorbide (DMI) or N-alkyl-2-pyrrolidone (e.g. N-butyl-2-pyrrolidinone), and the second solvent is selected from acetone; dimethyl sulfoxide (DMSO); dimethyl isosorbide (DMI); (+)-limonene; cyrene; methyl oleate; triethyl phosphate (TEP); γ-butryolactone; 1,2-butylene carbonate; methyl ethyl ketone (MEK); ‘palm oil’; cyclopentanone; dioctyl adipate; isophorone; acetyl triethyl citrate (ATEC); oleic acid; ethyl lactate; methyl lactate, dimethylsulfoxide, γ-butyrolactone; acetyl tributyl citrate; triethylcitrate; triethylene glycol diacetate; triethyleneglycol diacetate acetone; γ-valerolactone; ethyl acetate; propan-2-ol (IPA), ethanol, methyl 5-(dimethylamino)-2-methyl-5-oxopentanoate (e.g. Rhodiasolv® Polarclean (CAS-No. 174627-68-9)) and N-butyl-2-pyrrolidinone (e.g. TamiSolve® NxG), or mixtures thereof. The non-toxic solvent may comprise the first solvent and second solvent present in an amount of about 5:95 to about 95:5 (e.g. about 10:90 to about 90:10), more preferably about 20:80 to about 80:20, and even more preferably about 25:75 to about 75:25. For example, the non-toxic solvent may comprise the first solvent and second solvent in a wt. % ratio of 30:70, 35:65, 40:60, 45:55, 50:50, 55:45, 60:40, 65:35, 70:30. Preferably, the wt. % ratio of the first solvent and second solvent is about 25:75, 50:50 or 75:25.

The polymer may be any polymer suitable for use for tissue engineering.

In some examples, the polymer may comprise cellulose or cellulosic polymers or mixtures of both, polysulfone, polypropylene, acrylonitrile butadiene styrene (ABS), polycarbonate, polyethylene, or mixtures thereof. Suitable polymers include biodegradable polymers. Biodegradable polymers are any polymers that may be broken down by biological systems, such as polymers that can be broken down into harmless products by the action of living organisms.

In some examples, the polymer comprises one or more of a polystyrene (e.g. high impact polystyrenes), polycaprolactone, polysulfone, cellulose acetate, cellulose triacetate, polyethersulfone, acrylonitrile butadiene styrene, sulfonated polymers, polymethyl methacrylate, and/or derivatives of lactic and glycolic acid (e.g. poly(lactic acids), poly(l-lactic acid) (PLLA), poly(d-lactic acid) poly(glycolic acid), poly(lactic-co-glycolic acid) and polyhydroxyalkanoates).

In some examples, the polymer comprises one or more of a polystyrene (e.g. high impact polystyrenes), polycaprolactone, polysulfone, cellulose acetate, cellulose triacetate, polyethersulfone, acrylonitrile butadiene styrene, sulfonated polymers, polymethyl methacrylate, poly(lactic acid) (PLA), poly(lactic-co-glycolic acid) (PLGA), and/or polyhydroxyalkanoates (PHA).

In some examples, the polymer is a non-fluorinated polymer.

In some examples, the polymer comprises a non-sulfonated polymer.

For example, the polymer comprises one or more of a polystyrene (e.g. high impact polystyrenes), polycaprolactone, polysulfone, cellulose acetate, cellulose triacetate, polyethersulfone, acrylonitrile butadiene styrene, sulfonated polymers, polymethyl methacrylate, poly(lactic acid) (PLA), poly(lactic-co-glycolic acid) (PLGA), and/or polyhydroxyalkanoates (PHA).

In some examples, the polymer comprises or consists of a polystyrene.

In some examples, the polymer comprises or consists of a high impact polystyrene.

In some examples, the polymer comprises or consists of polycaprolactone.

In some examples, the polymer comprises or consists of polysulfone.

In some examples, the polymer comprises or consists of cellulose acetate.

In some examples, the polymer comprises or consists of cellulose triacetate.

In some examples, the polymer comprises or consists of polyethersulfone.

In some examples, the polymer comprises or consists of acrylonitrile butadiene styrene.

In some examples, the polymer comprises or consists of a poly(lactic acid) or derivatives thereof. In some examples, the polymer comprises or consists of a poly(lactic acid).

In some examples, the polymer comprises or consists of a poly(glycolic acid) or derivatives thereof. In some examples, the polymer comprises or consists of a poly(glycolic acid).

In some examples, the polymer comprises or consists of a poly(lactic-co-glycolic acid) or derivatives thereof. In some examples, the polymer comprises or consists of a poly(lactic-co-glycolic acid).

In some examples, the polymer comprises or consists of polyhydroxyalkanoate.

In some examples, the polymer comprises or consists of polymethyl methacrylate.

The polymer may be a biocompatible polymer. Biocompatible polymers are, along with any metabolites or degradation products thereof, generally non-toxic to cells or to a recipient (such as a human or animal), and do not cause any significant adverse effects to cells or a recipient, at concentrations resulting from the degradation of the polymers. Generally speaking, biocompatible polymers are polymers that do not elicit result in negative effects on cell health or in a recipient. As one use for the hollow fibres described herein is the production of comestible products, biocompatible and/or biodegradable polymers may be advantageous if the scaffold or part thereof is consumed (for example ingested) by a person.

Biodegradable polymers include linear aliphatic polyesters such as polylactic acid, polyglycolic acid, polycaprolactone, polyhydroxybutyrate, polyhydroxyvalerate and their copolymers within the aliphatic polyester family such as poly(lactic-co-glycolic acid) and poly(glycolic acid-co-caprolactone); copolymers of linear aliphatic polyesters and other polymers such as poly(glycolic acid-co-trimethylene carbonate) copolymers, poly(lactic acidco-lysine) copolymers, tyrosine-based polyarylates or polyiminocarbonates or polycarbonates, poly(lactide-urethane) and poly(ester-amide) polymers; polyanhydrides such as poly(sebacic anhydride); polyorthoesters such as 3,9-diethyidiene-2,4,8,10-tetraoxaspiro5,5-undecane based polymers; poly(ester-ether) such as poly-p-dioxanone; polyamides, poly(amide-enamines) and poly(amido amine) dendrimers; and phosphorus-based polymers such as polyphosphazene and poly [bis(carboxy-lactophenoxy)] phosphazene.

The polymers may be edible polymers. Edible polymers refers to any polymer that is acceptable for use in an edible product. Examples of edible polymers include polyvinyl alcohol, carboxyvinyl polymer, hydroxypropylmethylcellulose, hydroxyethylcellulose, methylcellulose, ethylcellulose, lowsubstituted hydroxypropylcellulose, crystalline cellulose, carboxymethylcellulose sodium, a synthetic polymer compound such as carboxymethylcellulose calcium, carboxymethylcellulose and carboxymethylstarch sodium, sodium alginate, dextran, casein, pullulan, pectin, guar gum, xanthan gum, tragacanth gum, acacia gum, zein, gelatin, chitin and chitosan, silk, fibrin and polymer compounds obtained from natural products such as starch or soybean. Edible polymers may include edible proteins from animal or plant sources. For example such edible proteins may be derived from legumes such as chickpea or pea. Edible polymers may also be derivable from waste food such as potatoes. In some examples, the edible polymer comprises zein. In some examples, the polymer may be a mixture of an edible polymer and second polymer.

The polymer may be an aliphatic polyester such as polycaprolactone, polyesteramides, modified polyethylene terephthalate, polylactic acid (PLA) and its copolymers, terpolymers based on polylactic acid, polyglycolic acid, polyalkylene carbonates (such as polyethylene carbonate), polyhydroxyalkanoates (PHA), poly-3-hydroxybutyrate (PHB), poly-3-hydroxyvalerate (PHV), poly-3-hydroxybutyrate-co-4-hydroybutyrate, poly-3-hydroxybutyrate-co-3-hydroxyvalerate copolymers (PHBV), poly-3-hydroxybutyrate-co-3-hydroxyhexanoate, poly-3-hydroxybutyrate-co-3-hydroxyoctanoate, poly-3-hydroxybutyrate-co-3-hydroxydecanoate, poly-3-hydroxybutyrate-co-3-hydroxyoctadecanoate, and succinate-based aliphatic polymers (e.g., polybutylene succinate, polybutylene succinate adipate, polyethylene succinate, etc.); aromatic polyesters and modified aromatic polyesters; and aliphatic-aromatic copolyesters.

Preferably, the polymer is selected from the group consisting of polystyrene, polycarbonate, polypropylene, polyvinylidene fluoride, polycaprolactone, polysulfone, cellulose acetate, cellulose triacetate, polyethersulfone, polyphenylenesulfon, polymethyl methacrylate, acrylonitrile butadiene styrene or mixtures thereof. In some examples, if two or more polymers are present, the wt. % ratio of the first polymer and the second polymer is about 5:95, 10:90, 15:85, 20:80, 25:75, 30:70, 35:65, 40:60, 45:55, 50:50, 55:45, 60:40, 65:35, 70:30, 75:25, 80:20, 85:15, 90:10 or 95:5. In some embodiments the polymer may act as a low functional inclusion additive and be present in amount less than 5 wt %.

In some examples, the polymer comprises polystyrene. The polymer can comprise polystyrene and a second polymer. The second polymer can be selected from the group consisting of polycarbonate, polypropylene, polyvinylidene fluoride, polycaprolactone, polysulfone, cellulose acetate, cellulose triacetate, polyethersulfone, polyphenylenesulfon, polymethyl methacrylate, acrylonitrile butadiene styrene or mixtures thereof. The composition can comprise polystyrene and a second polymer in a wt. % ratio of about 5:95, 10:90, 15:85, 20:80, 25:75, 30:70, 35:65, 40:60, 45:55, 50:50, 55:45, 60:40, 65:35, 70:30, 75:25, 80:20, 85:15, 90:10 or 95:5.

In some examples, the polymer comprises an edible polymer as described herein and polystyrene. For example, the polymer may comprise zein and polystyrene.

In some examples, the polymer comprises polysulfone. The polymer can comprise polysulfone and a second polymer. The second polymer can be selected from the group consisting of polystyrene, polycarbonate, polypropylene, polyvinylidene fluoride, polycaprolactone, cellulose cellulose acetate, triacetate, polyethersulfone, polyphenylenesulfon, polymethyl methacrylate, acrylonitrile butadiene styrene or mixtures thereof. The composition can comprise polysulfone and a second polymer in a wt. % ratio of about 5:95 to about 95:5 (e.g. about 10:90 to about 90:10), more preferably about 20:80 to about 80:20, and even more preferably about 25:75 to about 75:25. Polysulfone and the second polymer may be present in a wt. % ratio of 30:70, 35:65, 40:60, 45:55, 50:50, 55:45, 60:40, 65:35, 70:30.

Preferably, the polymer comprises polystyrene and/or polysulfone. Preferably, the polymer comprises polystyrene and polysulfone in a wt. % ratio of about 5:95 to about 95:5 (e.g. about 10:90 to about 90:10), more preferably about 20:80 to about 80:20, and even more preferably about 25:75 to about 75:25. Polystyrene and polysulfone may be present in a wt. % ratio of 30:70, 35:65, 40:60, 45:55, 50:50, 55:45, 60:40, 65:35, 70:30. Preferably, the composition comprises about 80 wt. % of polystyrene and about 20 wt. % of polysulfone, or about 60 wt. % of polystyrene and about 40 wt. %.

In some examples, the polymer comprises high impact polystyrene (HIPS). The polymer can comprise HIPS and a second polymer. The second polymer can be selected from the group consisting of polystyrene, polycarbonate, polypropylene, polyvinylidene fluoride, polycaprolactone, polysulfone, cellulose acetate, cellulose triacetate, polyethersulfone, polyphenylenesulfon, polymethyl methacrylate, acrylonitrile butadiene styrene or mixtures thereof. The composition can comprise HIPS and a second polymer in a wt. % ratio of about 5:95 to about 95:5 (e.g. about 10:90 to about 90:10), more preferably about 20:80 to about 80:20, and even more preferably about 25:75 to about 75:25. HIPS and the second polymer may be present in a wt. % ratio of 30:70, 35:65, 40:60, 45:55, 50:50, 55:45, 60:40, 65:35, 70:30.

The polymer may be present in the composition in an amount of from about 5 wt. % to about 50 wt. %, preferably from about 10 wt. % to about 40 wt. %, from about 15 wt. % to about 30 wt. %.

The wt. % ratio of the non-toxic solvent and the polymer present in the composition may vary depending on the components present and solubility of the polymer thereof. In some examples, the polymer and non-toxic solvent may be present in the composition in a wt. % ratio of about 1:1 to 10, preferably about 1:2 to 7, more preferably about 1:3 to 6.

In some examples, the polymer is selected from polystyrene and/or polysulfone and the non-toxic solvent comprises N-butyl-2-pyrrolidinone, dimethyl isosorbine, acetone or mixtures thereof. For example, the non-toxic solvent can comprise N-butyl-2-pyrrolidinone and/or acetone. Alternatively, the non-toxic solvent can comprise dimethyl isosorbine and/or acetone.

In some examples, when the non-toxic solvent comprises N-alkyl-2-pyrrolidone (e.g. N-butyl-2-pyrrolidinone), the polymer is selected from polystyrene, acrylonitrile butadiene, polysulfone, high impact polystyrene (HIP), or mixtures thereof. In some examples, the non-toxic solvent comprises N-butyl-2-pyrrolidinone and the polymer is high impact polystyrene (HIPS). In some examples, the non-toxic solvent comprises N-butyl-2-pyrrolidinone and the polymer is polystyrene.

In some examples, when the non-toxic solvent comprises dimethyl isosorbide, the polymer is selected from polystyrene, acrylonitrile butadiene, polysulfone, high impact polystyrene (HIPS), or mixtures thereof. HIPS is a rubberized polystyrene modified with polybutadiene. In some examples, the non-toxic solvent comprises N-butyl-2-pyrrolidinone and the polymer is polystyrene.

In some examples, when the non-toxic solvent comprises methyl 5-(dimethylamino)-2-methyl-5-oxopentanoate, the polymer is selected from polystyrene, acrylonitrile butadiene, polysulfone, high impact polystyrene (HIPS), or mixtures thereof. In some examples, the non-toxic solvent comprises N-butyl-2-pyrrolidinone and the polymer is polystyrene.

As used herein, the term ‘porogen’ means a component capable of improving pore connectivity and enhancing pore formation e.g. suitable porogens typically swell in water. Typically, the porogen should solubilise in the non-solvent. Preferably, the porogen is non-toxic, biodegradable and/or biocompatible component (e.g. it is generally non-toxic to cells or to a recipient (such as a human or animal), and does not cause any significant adverse effects to cells or a recipient, at concentrations resulting from degradation). Preferably, the porogen and the polymer are not the same component.

The porogen can be present in the composition in an amount of less than 60 wt. %, preferably less than 30 wt. %, more preferably less than 15 wt. %. In some examples, the porogen is present in the composition in an amount of less than 10 wt. %, more preferably less than 8 wt. %. The porogen is present in an amount of about 0.01 wt. % to about 60 wt. % (e.g. about 0.01 wt. % to about 60 wt. %, preferably about 1 wt. % to about 30 wt. %, more preferably about 5 wt. % to about 15 wt. %, even more preferably about 7 wt. % to about 11 wt. %). In some examples, the porogen is present in an amount of about 10 wt. % of the composition.

The polymer and the porogen can be present in the composition in a ratio of about 1:0.01 to 10 (e.g. about 1:0.5 to 5). Preferably, the polymer and porogen are present in a ratio of about 1:0.1 to 3 (e.g. about 1:2), even more preferably 1:0.1 to 1.5 (e.g. about 1:1.5) In some examples, the polymer and the porogen are present in the composition in a ratio of about 1:0.3 to 1.2, more preferably about 1:0.45 to 1.1.

The porogen may include, but is not particularly limited to, the following components: salt particles, polymers, soluble starches, marine polymers, cellulose (e.g. fibres, micro/nanocrystalline cellulose, cellulose nanofibre), nylon, glass, silica, surfactant, sugars and sugar alcohols. Although any suitable component capable of swelling in water can be used as a porogen according to the present disclosure. A wide variety of porogens are known in the art, for example as noted in Lakshmi et al—“Trends in porogens toward material fabrication: Recent progresses and challenges. Polymers 2022, 14, 5209. Examples of organic and inorganic porogens are found in tables below as noted in the publication by Lakshmi et al:

TABLE 1
Various types of porogens with their functions and applications.

| S. No. | Porogens Used | Functions | Applications |
|---|---|---|---|
| 1. | Water-soluble polymers such as | Increases mean pore size | Water filtration [symbol] Pharmaceutical |
| | Polyethylene glycol (PEG), | distribution, membrane porosity | and biomedical applications, |
| | poly(vinylpyrrolidene) (PVP), | and hydrophilicity. | fuel cell applications, etc. |
| | Polyvinyl alcohol (PVA), etc. | | |
| 2. | Low molecular weight Inorganic | Good adsorption | Enhanced heterogeneous |
| | salts, Lithium chloride, Zinc | capabilities, high | photocatalysts and their |
| | chloride, SiO₂, etc. | transparency, easy regeneration | application to various reactions |
| | | | for organic pollutant removal |
| | | | from air and water |
| 3. | Calcium Carbonate | Improved the apparent porosity | High apparent |
| | | and enlarged the pore size with | porosity ceramics |
| | | good mechanical strength | |
| 4. | Ammonium bicarbonate | Enhanced porosity and show | Shape memory alloys |
| | | more uniform pore distribution | Highly open porous |
| | | Well interconnected macroporous | biodegradable scaffolds for |
| | | scaffolds were produced having | tissue regeneration [symbol] |
| | | mean pore diameters of around | |
| | | 300-400 μm | |
| 5. | Waste biological resource such as | Reduction composition of pore | Microfiltrations and microbial |
| | saw dust, potato, wheat, corn, rice | former results in reduction in | filtration applications. |
| | starch, etc. | membrane pore size and porosity. | Burners, anodes, thermal barrier |
| | Corncob bio char from K₂FeO₄ | Porosity also depends on | coatings and insulating layers. |
| | and KOH | thermal conductivity | Capacitive deionization |
| | | Good capacitance equivalent to | plate materials or electrodes |
| | | graphene materials | |
| 6. | Fine clay bricks-wastes from | Porosity, water absorption, | Innovative building materials |
| | renewable or mineral resources | density, mechanical resistance and | |
| | | even thermal | |
| | | insulation is enhanced | |
| | | and modified. | |
| 7. | Carbon black | Pores were helpful for enhancing | Alumina porous ceramics |
| | Activated carbon from the | the strength and decreasing the | Proton exchange membrane |
| | palm oil shell | thermal conductivity | fuel cells |
| | | Pore [symbol] increase the porosity | P [symbol] water microfiltration |
| | | and pore volume | |
| | | Shown best | |
| | | membrane permeability | |
| 8. | Naphthalene, carbon beads or fibers | Porosities up to 90% with pores | Drug delivery and |
| | polymers such as PMMA | ranging from 1 to 2000 mm in size | manufacturing of 3D scaffolds |
| | (polymethylmethaacrylate), | are reported | with desired porosity |
| | polyurethane, cellulose and | | |
| | paraffin oil | | |
| 9. | Biodegradable polymer: Chitosan | Produced anodes with lower | Solid oxide fuel cells |
| | | fracture strength and modest | |
| | | electrical conductivity | |
| 10. | Marine source seaweed | Continuous porous structure with | Membranes for |
| | polysaccharides: κ-Carrageenan | uniformly distributed pores was | various applications |
| | | obtained. It also increased the | |
| | | membrane porosity and mean | |
| | | pore diameter increased | |
| 11. | Marine source seaweed | Showed a high porosity and an | 3D Porous hydrogel as |
| | polysaccharides: Alginate | open porous structure | meniscus substitute |
| 12. | Marine source seaweed | Very high influence on the | Ultrafiltration membranes |
| | polysaccharides: Ulvan | efficiency and | |
| | | morphological properties | |

[symbol]

indicates data missing or illegible when filed

TABLE 2
Different inorganic nanoparticles used in thin film nanocomposite membranes.

| S. No. | Nature of the Membranes | Inorganic Porogens Used | Properties Tuned | Applications |
|---|---|---|---|---|
| 1. | Thin-film nanocomposite | SiO₂ nanoparticles | Higher water | Forward osmosis [9] |
| 2. | (TFN) membranes | Amine functionalized | permeability, high | Forward osmosis [10] |
| | | multi-walled carbon | water flux and better | |
| | | nanotubes (F-MWCNTs) | salt rejection | |
| 3. | | TiO₂ nanoparticles | Higher water | Forward osmosis [11] |
| | | | permeability and low | |
| | | | reverse solute flux | |
| 4. | | Porous zeolite | Higher water | Forward osmosis [12] |
| | | nanoparticles | permeability and high | |
| 5. | | NaY zeolite nanoparticles | water flux | Forward osmosis [13] |
| 6. | Metal matrix membranes | Silica gel | Higher water | Forward osmosis [14] |
| | | | permeability, high | |
| | | | water flux and better | |
| | | | salt rejection | |

In some examples, the porogen is or includes salt particles, polymers, soluble starches, marine polymers, cellulose (e.g. fibres, micro/nanocrystalline cellulose, cellulose nanofibre), polyvinyl alcohol (PVOH), polyvinylpyrrolidone (PVP), surfactant, sugars and/or sugar alcohols.

Examples of sugars include, but are not limited to glucose, fructose, sucrose, maltose, galactose and trehalose. Examples of sugar alcohols (polyols) include but are not limited to, glycerol, threitol, xylitol, erythritol, mannitol, sorbitol, maltitol, erythritol, and lactitol.

In some examples, the porogen is or comprises polyvinyl alcohol (PVOH).

In some examples, the porogen comprises salt particles (e.g. microcrystalline salt particles). Salt particles may be prepared by any conventional known method. The salt particles may include sodium chloride.

In some examples, the porogen comprises polyethylene glycol (PEG) (herein also referred to as ‘polyethylene oxide (PEO)’. Preferably, the PEG/PEO has a number average molecular weight of about 300 g/mol or more (e.g. about 300 to about 500,000 g/mol). Suitable number average molecular weight of PEG includes, but is not particularly limited to, PEG 100, PEG 200, PEG 300, PEG 400, PEG 600, PEG 1000, PEG 1500, PEG 2000, PEG 3000, PEG 3500, PEG 4000, PEG 5000, PEG 6000, PEG 8000, PEG 10,000, PEG 20,000. Suitable number average molecular weight of PEO includes, but is not particularly limited to, PEO 25,000, PEO 30,000, PEO 35,000, PEO 40,000, PEO 45,000, PEO 50,000, PEO 55,000, PEO 100,000, PEO 100,000, PEO 200,000, PEO 300,000, PEO 400,000, PEO 500,000, PEO 600,000, PEO in +100,000 increments up to 5,000,000.

In some examples, the porogen is polyethylene glycol (PEG). More preferably, the porogen is PEG 400. In some examples, the porogen (e.g. PEG) is present in the composition in an amount of 0 wt. % to about 60 wt. %, e.g. about 0.01 wt. % to about 60 wt. %, preferably about 1 wt. % to about 30 wt. %, more preferably about 5 wt. % to about 15 wt. %, even more preferably about 7 wt. % to about 11 wt. % (e.g. about 10 wt. %) of the composition.

In some examples, the porogen comprises polyvinylpyrrolidone (PVP). Polyvinylpyrrolidone (also referred to as PVP) refers to a water soluble polymer compound obtained by polymerizing N-vinyl-2-pyrrolidone (N-vinyl-2-pyrrolidone). In some examples, the porogen comprises PVP-10. PVP-10 refers to a PVP polymer having an average molecular weight of 10,000 Da.

In some examples, the sum of total weight of the non-toxic solvent, polymer and porogen is 95 to 100%, preferably 97 to 100%, more preferably 98 to 100% of the total weight of the composition. Preferably, the sum of total weight of the non-toxic solvent, polymer and porogen is 100% of the total weight of the composition.

In some examples, the method further comprises subjecting the hollow fibres to post-synthesis surface modification. For example, post-synthesis chemical surface modification, coating and/or plasma modification. In some examples, the method further comprises subjecting the hollow fibres to post-synthesis plasma modification. In some examples, the method further comprises subjecting the hollow fibres to post-synthesis coating. In some examples, the method further comprises subjecting the hollow fibres to post-synthesis chemical surface modification. Plasma modification refers to a technique of modifying the surface of a material with high-energy plasma. Plasma may be formed by ionizing a gas, for example by the application of energy to the gas. Gases that may be used for plasma modification include but are not limited to oxygen, nitrogen, hydrogen or carbon dioxide. Chemical surface modification refers to altering the surface of the hollow fibres by exposing the surface of the hollow fibres to one or more chemicals. For example, chemical surface modification may include chemical grafting (i.e. via a chemical reaction) of acrylic acid and/or methacrylate species onto the surface of the hollow fibres. For example, chemical modification may include chemical grafting of acrylic acid (AA), ethylene glycol methacrylic phosphate (EMP) and/or N-hydroxymethylacrylamide (HMA) onto the surface of the hollow fibre.

In some examples, the surface modification increases the hydrophilicity of the surface. For example, hydrophobicity or changes thereof may be determined by analysis of the contact angle of fluid on the surface of the hollow fibres. For example, the surface modification may reduce a contact angle of a fluid on the surface of the hollow fibres. Methods of determining hydrophobicity are known in the art. In some examples, the surface modification comprises contacting the fibres with an oxygen plasma. Methods of carrying coating include but are not limited to dipping or spraying of a coating onto the surface.

According to a second aspect of the present disclosure, there is provided a composition for forming a hollow fibre for tissue engineering. The composition comprises:
a polymer;
a porogen; and
a non-toxic solvent.

The polymer can be a polymer according to the first aspect described herein. The porogen can be a porogen according to the first aspect described herein. The non-toxic solvent can be a non-toxic solvent according to the first aspect described herein. Preferably, the composition is used in the method according to the first aspect described herein. Preferably, the composition is suitable for forming a porous hollow fibre membrane.

Provided herein is a hollow fibre, obtainable by (e.g. obtained directly from) a method according to the first aspect described herein and/or obtainable from (e.g. obtained directly from) a composition according to the second aspect described herein. Preferably, the hollow fibre is obtained by (e.g. obtained directly from) a method according to the first aspect described herein.

The hollow fibre is preferably a porous hollow fibre (e.g. a porous hollow fibre membrane) having suitable characteristics for applications in tissue engineering. However, it would be understood by a person skilled in the art that the calculations may apply to other hollow fibres prepared according to the method of the present disclosure.

A partial amount of the non-toxic solvent may be present in the hollow fibre, herein referred to as residual solvent. Typically, residual solvent may remain entrapped in the hollow fibre. In some examples, the method comprises a further step of washing the hollow fibre (e.g. to remove residual solvent). Preferably, the hollow fibre comprises no more than 10 wt. % of residual solvent. In some examples, the hollow fibre comprises less than 10 wt. % (e.g. less than 5 wt. %) of residual solvent. Preferably, the hollow fibre comprises less than 4 wt. %, less than 3 wt. %, less than 2 wt. %, less than 1 wt. %, less than 0.75 wt. %, less than 0.5 wt. %, less than 0.25 wt. %, less than 0.1 wt. %, less than 0.05 wt. % of residual solvent present.

Advantageously, the present disclosure provides an improved method whilst mitigating the use of toxic solvents (e.g. cytotoxic solvents, CMR solvents, solvents that pose serious risk for human health). Thus, also reducing the number of washes required to remove residual solvent. Typically, use of toxic solvents results in large amounts of aqueous waste streams and high energy inputs required to separate the fibres and remove or recover the solvents. The present disclosure, therefore, aims to mitigate such issues relating to damage caused to human health and the environmental impacts, for example relating to the manufacturing method or post-manufacturing uses which can lead to negative impacts on operational safety and costs. Thus mitigating the use of toxic solvents provides hollow fibres suitable for culturing cells (in other words, not hazardous for culturing cells).

The hollow fibre is preferably porous. In some examples, the hollow fibre has an average pore diameter from about 0.001 μm to about 100 μm, preferably having an average pore diameter from about 0.01 μm to about 50 μm. More preferable, the average pore diameter is from about 0.03 μm to about 20 μm, from about 0.05 μm to about 10 μm, from about 0.1 μm to about 5 μm, or from about 0.3 μm to about 2 μm. In some examples, the average pore diameter is at least 0.1 μm. Methods of determining average pore diameter are well known in the art and include, microscopy based analysis such as scanning electron microscopy (SEM) image analysis or atomic force microscopy methods. In some examples, average pore diameter is determined by SEM. For example, by analysis of the hollow fibre surface using SEM image analysis. For example methods of determining pore diameter, see Widiatmoko, Eko, Mikrajuddin Abdullah, and Khairurrijal. “A method to measure pore size distribution of porous materials using scanning electron microscopy images.” Aip conference proceedings. Vol. 1284. No. 1. American Institute of Physics, 2010; Nakao, Shin-ichi. “Determination of pore size and pore size distribution: 3. Filtration membranes.” Journal of Membrane Science 96.1-2 (1994): 131-165; and Nakao, Shin-ichi. “Determination of pore size and pore size distribution: 3. Filtration membranes.” Journal of Membrane Science 96.1-2 (1994): 131-165 all of which are incorporated herein by reference.

It is noted that concentrations of components (e.g. polymer, solvent and porogen) provided herein as wt. % may be determined by dividing the mass of a component by the total mass of the entity which is the component is comprised (e.g. composition, hollow fibre or membrane). For example, 0.5 grams of membrane including 0.05 grams of porogen and 0.45 grams of polymer has 10 wt. % porogen and 90 wt. % polymer. For example, 100 grams of a dope solution formulation (composition) including 17.5 grams of polymer and 10 grams of porogen dissolved in 72.5 grams of solvent has 17.5 wt. % polymer and 10 wt. % porogen dissolved in 72.5 wt. % solvent.

The hollow fibre can be used in tissue engineering scaffolds. In some examples, the hollow fibre is used in an application of cell proliferation, cell differentiation and/or culturing or harvesting of cells.

According to a third aspect of the present disclosure, there is provided a method of culturing cells. The method comprises providing one or more hollow fibres obtainable by a method according to the first aspect described herein or obtainable from a composition according to the second aspect described herein, and using the one or more hollow fibres in a method of culturing cells.

The method could comprise the step of seeding cells onto the one or more hollow fibres. The method further comprises subjecting the one or more hollow fibres to conditions suitable to allow initial attachment of the cells to an outer surface of each of the one or more hollow fibres, and culturing the cells attached to the outer surface of each of the one or more hollow fibres under conditions suitable for proliferation and/or differentiation of the cells.

The one or more hollow fibres may be a plurality of hollow fibres. A fluid (e.g. cell culture media) may be introduced into the channels of the hollow fibres. In use for culturing cells, the cells may be cultured on the exterior of the hollow fibres and cell culture media (e.g., nutrients) can be circulated through the channels of the hollow fibres to facilitate cell propagation and other processes. In particular, the outer surfaces of the hollow fibres provide a surface area for cell attachment. In other examples, the inner surfaces of the hollow fibres may provide a surface area for cell attachment. In other examples, the inner and outer surfaces of the hollow fibres may provide a surface area for cell attachment. The hollow fibres are preferably porous and semi-permeable. The hollow fibres preferably have pores sized to retain both cells and cell-secreted products, but permit movement of cell culture media (e.g., nutrients) through the walls of the hollow fibres. In this way, during use, cells can adhere to the outer and/or inner surfaces of the hollow fibres and cell culture media can be passed through the channels of the hollow fibres to support cell culturing.

Preferably, the cells are muscle cells. The muscle cells can be derived from at least one comestible animal cell. For example, the muscle cells may comprise one or more of fibroblasts, skeletal muscle cells, smooth muscle cells, and/or myoblasts. Preferably, the muscle cells are derived from one or more of non-human embryonic stem cells and/or pluripotent stem cells.

A comestible product comprising muscle cells may be obtained by the method according to the third aspect described herein. A comestible product (e.g. meat analogues/a cultured meat product) may be obtainable by the method according to the third aspect described herein. Preferably, the comestible product comprises muscle cells.

### EXAMPLES

The present invention will now be described in further detail by the examples provided below. The following definitions of terms and determination methods apply for the above general description of the invention as well as to the below examples unless otherwise defined.

### Measurement Methods

### Wetting Behaviour/Contact Angle Measurement

The wetting behaviour of the fibres was analysed by measuring the water contact angle (static water contact or Sessile drop) using a goniometer (OCA20, DataPhysics Instruments, Germany) at room temperature. A drop of 0.5 μL water was placed on the membrane surface and the average of 10 measurements reported. Image analysis software was used to extract droplet and surface profiles and static contact angles were calculated via Young's equation.

### Scanning Electron Micrograph (SEM)

The surface and cross-section of the flat sheet/hollow fibres were analysed using a scanning electron microscope (SEM, JEOL SEM6480LV, Japan). Membrane samples were first dried under vacuum (~40 °C) overnight and then sputter-coated with a 10 nm thickness of chromium (Edwards Sputter Coater S150B, Mechatech Systems, UK) for SEM imaging. Analysis of SEM micrographs were carried out using Image J.

### Surface and Cross-Section

The surface and cross-section of the hollow fibres were analysed using a scanning electron microscope (SEM, JEOL SEM6480LV, Japan). Membrane samples were first dried under vacuum (~40 °C) overnight and then sputter-coated with a 10 nm thickness of chromium (Edwards Sputter Coater S150B, Mechatech Systems, UK) for SEM imaging.

### Filtration Performance, Average Permeance

The filtration performance of the fibres was evaluated by using a cross-flow filtration set up at room temperature. The experiments were carried out in glass modules with ~6 cm long containing single hollow fibre. The permeance, K, (LMH bar⁻¹) of the membrane was calculated by using the following equation:

$$K = \frac{V}{Δ t \cdot A \cdot Δ P}$$

wherein
   V is the volume of permeate collected (m³) over time Δt (hr);
   A is the effective fibre area (m²);
   Δp is the transmembrane pressure (bar).

All filtration experiments were carried out at 0.5 bar constant pressure and repeated three times. The total sum of the non-toxic solvent, polymer and porogen in the Examples provided herein is equal to 100 wt. %.

### Suitable Conditions

Conditions for mixing the composition (e.g. solubilising the polymer in the solvent) are not particularly limited, and, for example, they may be carried out at room temperature or may be carried out by heating. The heating conditions are appropriately selected depending on the type of polymer. Preferably, the heating temperature is between room temperature to around 50 °C, for example, 18 to 50 °C, preferably 40 °C. Typically, the conditions may include atmospheric pressure in a sealed vessel.

Conditions for the coagulation bath (e.g. coagulation bath temperatures) are not particularly limited. The temperature is are appropriately selected depending on the composition extruded. The temperature can range between 0 to 100 °C, for example, temperatures can be low (at around 3 to 5 °C) or high ((at around 50 to 59 °C).

### Example 1: Method of Forming a Hollow Fibre

Using PolarClean® (PC) (as the non-toxic solvent), polystyrene (PS) (17.5 wt %) and polyethylene glycol (PEG, 10 wt. %) were first dissolved in PC solvent and then stirred using a roller mixer (120 VAC, Cole-Parmar) for 3 days at room temperature to prepare a dope solution.

The dope solution was then left overnight to de-gas. Dry-wet spinning technique was used to fabricate hollow fibres by using a spinning rig. The spinning rig was first set up in place and the bubble-free dope solution was poured into the spinning tank. The tank was pressurised using nitrogen (N₂) gas at the desired pressure (0.2 bar) to extrude the dope solution through the spinneret. The air gap (10 cm) was adjusted by adjusting the coagulation bath using a hydraulic jack.

R.O. water was used as a nonsolvent and bore liquid at room temperature at a flow rate of 12 (ml min⁻¹). The resulting fibres were left at the bottom of the coagulation bath. For the removal of PC, the fibres were kept in water for at least 3 days and the water in the bath was replaced with the fresh water every 24 hours.

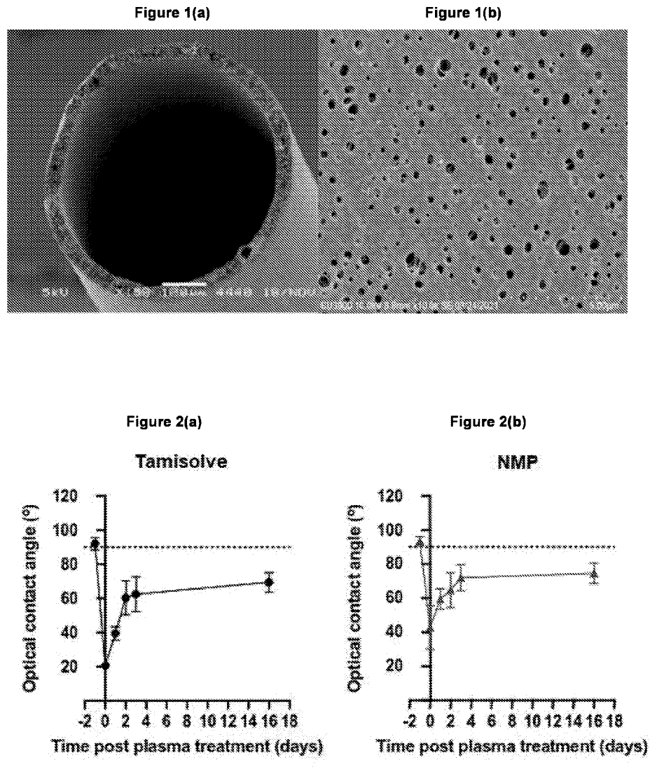

The hollow fibres produced had a wall thickness of approximately 55±5 μm having surface pore size range 0.5-1 μm, which suggest that fibres are in the order of microfiltration and therefore suitable for tissue engineering applications where cell size is typically between 10-20 μm. See FIGS. 1(a) and (b) which illustrate a (a) cross section and (b) surface of the hollow fibre obtained.

The hollow fibres showed pure water flux of ~1000 LMH at 0.5 bar, having porosity of ~55% and contact angle of ~15° after plasma treatment.

### Example 2: Compositions

Set out in Table 1 below is a (non-exhaustive) list of examples of a composition described herein (e.g. a composition used in the method according to the first aspect described herein). The wt. % amounts of solvent 1 and solvent 2 correspond to the amount present in the non-toxic solvent of the composition. The wt. % of the polymer corresponds to the amount present in the polymer of the composition. In Example numbers 2.1 to 2.33 the porogen is PEG400, wherein PEG400 is present in the composition in an amount of approximately 10 wt. %. In Examples 2.34 to 2.35, the porogen is NaCl, present in the composition in an amount of approximately 7 wt. %. The solubility of the polymer can be determined by conventional techniques (including visual confirmation).

TABLE 1

| | | Solvent 1 | | Solvent 2 | |
|---|---|---|---|---|---|
| No. | Solvent 1 | Wt. % | Solvent 2 | Wt. % | Temperature |
| | Polystyrene (100 wt. %) | | | | |
| 2.1 | Acetone | 50 | d-Limonene | 50 | R.T. |
| 2.2 | Acetone | 75 | d-Limonene | 25 | R.T. |
| 2.3 | d-Limonene | 100 | N/A | 0 | R.T. |
| 2.4 | Acetone | 25 | d-Limonene | 75 | R.T. |
| 2.5 | Acetone | 25 | d-Limonene | 75 | 40 °C |
| 2.6 | Acetone | 75 | d-Limonene | 25 | 40 °C |
| 2.7 | Acetone | 50 | d-Limonene | 50 | 40 °C |
| 2.8 | Acetone | 50 | Cyrene | 50 | 40 °C |
| 2.9 | d-Limonene | 100 | N/A | 0 | 40 °C |
| 2.10 | Dimethyl | 25 | d-Limonene | 75 | R.T. |
| | sulfoxide | | | | |
| | (DMSO) | | | | |
| 2.11 | Dimethyl | 100 | N/A | 0 | R.T. |
| | isosorbide | | | | |
| | (DMI) | | | | |
| 2.12 | Dimethyl | 100 | N/A | 0 | 40 °C |
| | isosorbide | | | | |
| | (DMI) | | | | |
| 2.13 | Acetone | 50 | Dimethyl | 50 | R.T. |
| | | | isosorbide | | |
| | | | (DMI) | | |
| 2.14 | Acetone | 75 | Dimethyl | 25 | R.T. |
| | | | isosorbide | | |
| | | | (DMI) | | |
| 2.15 | Acetone | 25 | Dimethyl | 75 | R.T. |
| | | | isosorbide | | |
| | | | (DMI) | | |
| 2.16 | Acetone | 25 | Dimethyl | 75 | 40 °C |
| | | | isosorbide | | |
| | | | (DMI) | | |
| 2.17 | Acetone | 75 | Dimethyl | 25 | 40 °C |
| | | | isosorbide | | |
| | | | (DMI) | | |
| 2.18 | Acetone | 50 | Dimethyl | 50 | 40 °C |
| | | | isosorbide | | |
| | | | (DMI) | | |
| 2.19 | Tamisolve | 100 | N/A | 0 | R.T. |
| 2.20 | Acetone | 75 | Tamisolve | 25 | R.T. |
| 2.21 | Acetone | 50 | Tamisolve | 50 | R.T. |
| 2.22 | Acetone | 25 | Tamisolve | 75 | R.T. |
| 2.23 | Dimethyl | 25 | Tamisolve | 75 | R.T. |
| | sulfoxide | | | | |
| | (DMSO) | | | | |
| 2.24 | Acetone | 75 | Tamisolve | 25 | 40 °C |
| 2.25 | Acetone | 50 | Tamisolve | 50 | 40 °C |
| 2.26 | Acetone | 25 | Tamisolve | 75 | 40 °C |
| 2.27 | Tamisolve | 100 | N/A | 0 | 40 °C |
| 2.28 | Dimethyl | 25 | Tamisolve | 75 | 40 °C |
| | sulfoxide | | | | |
| | (DMSO) | | | | |
| 2.29 | Dimethyl | 50 | Tamisolve | 50 | 40 °C |
| | sulfoxide | | | | |
| | (DMSO) | | | | |
| 2.30 | Polarclean | 100 | N/A | 0 | R.T. |
| | Polysulfone (100 wt. %) | | | | |
| 2.31 | Tamisolve | 100 | N/A | 0 | R.T. |
| | Acrylonitrile butadiene styrene (ABS) (100 wt. %) | | | | |
| 2.32 | Tamisolve | 100 | N/A | 0 | R.T. |
| | High impact polystyrene (HIPS) (100 wt. %) | | | | |
| 2.33 | Tamisolve | 100 | N/A | 0 | R.T. |
| | Polystyrene (80 wt, %) and polysulfone (20 wt. %) | | | | |
| 2.34 | Tamisolve | 100 | N/A | 0 | R.T. |
| | Polystyrene (60 wt. %) and polysulfone (40 wt. %) | | | | |
| 2.35 | Tamisolve | 100 | N/A | 0 | R.T. |

### Example 3: Flat Sheets

Set out in Table 2 below is a (non-exhaustive) list of compositions (e.g. a composition used in the method according to the first aspect described herein) from which a flat sheet was obtained.

Flat sheets were prepared by pouring a fixed mass (nominally 5 g) of dope solution onto a clean glass substrate and drawing the solution to a thickness of about 50 to 400 μm using a fixed gap casting bar) or casting knife across the slide. The resulting wet film was then placed into a 1 L coagulation bath. Water changes were made twice daily for 3 days (total of 6 L) to allow for solvent removal from the membrane

The wt. % amounts of solvent 1 and solvent 2 correspond to the amount present in the non-toxic solvent of the composition. The wt. % of the polymer corresponds to the amount present in the polymer of the composition. In Example numbers 3.1 to 3.4 and 3.6 to 3.13 the porogen is PEG400, wherein PEG400 is present in the composition in an amount of approximately 10 wt. %. In Example 3.5, the porogen is NaCl, present in the composition in an amount of approximately 7 wt. %.

TABLE 2

| | | Solvent 1 | | Solvent 2 | |
|---|---|---|---|---|---|
| No. | Solvent 1 | Wt. % | Solvent 2 | Wt. % | Temperature |
| | Polystyrene (100 wt. %) | | | | |
| 3.1 | Dimethyl | 100 | N/A | 0 | R.T. |
| | isosorbide | | | | |
| | (DMI) | | | | |
| 3.2 | Acetone | 25 | Dimethyl | 75 | R.T. |
| | | | isosorbide | | |
| | | | (DMI) | | |
| 3.3 | Dimethyl | 100 | N/A | 0 | 40 °C |
| | isosorbide | | | | |
| | (DMI) | | | | |
| 3.4 | Tamisolve | 100 | N/A | 0 | R.T. |
| 3.5 | Tamisolve | 100 | N/A | 0 | R.T. |
| 3.6 | Acetone | 50 | Tamisolve | 50 | R.T. |
| 3.7 | Acetone | 25 | Tamisolve | 75 | R.T. |
| 3.8 | Dimethyl | 25 | Tamisolve | 75 | R.T. |
| | sulfoxide | | | | |
| | (DMSO) | | | | |
| 3.9 | Tamisolve | 100 | N/A | 0 | 40 °C |
| 3.10 | Polarclean | 100 | N/A | 0 | R.T. |
| | Polysulfone (100 wt. %) | | | | |
| 3.11 | Tamisolve | 100 | N/A | 0 | R.T. |
| | Acrylonitrile butadiene styrene (ABS) (100 wt. %) | | | | |
| 3.12 | Tamisolve | 100 | N/A | 0 | R.T. |
| | High impact polystyrene (HIPS) (100 wt. %) | | | | |
| 3.13 | Tamisolve | 100 | N/A | 0 | R.T. |

### Example 4: Plasma Treatment Longevity

Plasma treatment longevity was shown by contact angle measurement (oxygen plasma) characterised as a function of time for the flat sheet membrane obtained from the composition of Example 3.4 (see FIG. 2(a)) in comparison to a flat sheet membrane obtained by replacing the non-toxic solvent of Example 3.4 with NMP (wt. 100%) as the solvent (see FIG. 2(b)). As can be seen in FIGS. 2(a) and (b), the membrane retains hydrophilicity after 16 days and plateaus.

It should be noted that Hydrophobic recovery appears to plateau (stabilise). All Day 16 OCA (optical contact angle) values are more hydrophilic than pre-plasma OCA values, i.e.

OCA_{Day 16}<OCA_{Day -1}
wherein Day−1=no plasma.

The Plasma treatment protocol was as follows:
1. Prepare samples to ensure they are dry and small enough to fit within the plasma chamber
2. Turn on the plasma chamber, press vent to release the door to the chamber, this can be removed to open the chamber
3. Wipe down the glass plate and internal chamber with tissue paper sprayed with 70% EtOH to clean the chamber, then leave to air dry (~5 minutes)
4. Turn on the oxygen supply: open the oxygen cylinder valve, open the regulator valve and set outlet pressure to 0.5 barg.
5. Set the plasma chamber power to 50 using the dial
6. Set the oxygen flow rate on the plasma chamber to ~10 NL/h using the rotameter.
7. Place your samples on the glass plate, insert it into the chamber, hold the door to the opening and press the pump button.
8. The oxygen flow rate will start to rise, close the rotameter slightly until it stabilises at ~10 NL/h.
9. Press the generate button. When a purple/blue plasma glow is seen start the time and wait 2 minutes. The oxygen flow rate will start to fluctuate, keep it in the range of ~0-20 NL/h.
10. When the 2 minute timer goes, press the generate button again to switch off the plasma generation. Wait approximately 30 seconds and then press the vent button to equalise the chamber pressure and open the door.
11. Remove your samples and place inside a sealed, clean container.

### Example 5: Cytocompatibility

The flat sheets obtained from the compositions of Examples, 3.1 (DMI), 3.2 (Ac/DMI 25/75), 3.4 (Tam), 3.6 (Ac/Tam 50/50), 3.7 (Ac/Tam 25/75) and 3.8 (DMSO/Tam 25/75) were plasma treated and then seeded with C2C12 cells. Commercial tissue culture plastic (TCP) was used as a comparison.

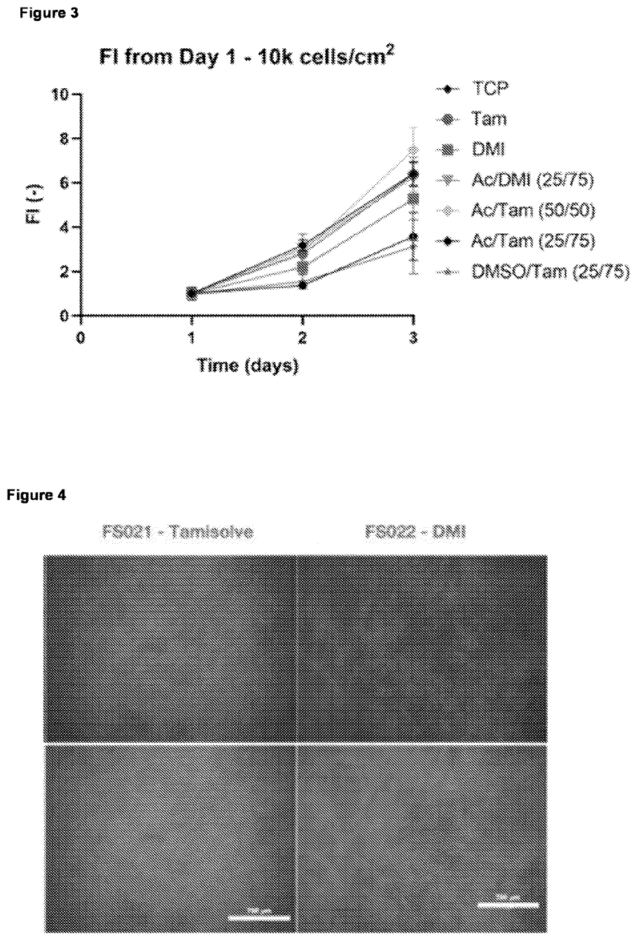

FIG. 3 shows the results of fold increase from Day 1 (10k cells/cm²). Fold increase (FI) is used as a method of normalising viable cell count. As can be seen, there was no overall toxicity exhibited, as there were viable cells and growth on all flat sheets (shown with the increasing fold increase of cells (FI) over time on the flat sheets:

FI=N(t)/N(t=24 h)

FIG. 4 shows live-dead cell staining images of the flat sheets obtained from the compositions of Examples 3.1 (DMI) and 3.4 (Tamisolve), after 7 days proliferation. As can be seen, the flat sheet obtained from the composition of Example 3.4 (Tamisolve) showed the surface covered with no dead cells visible and the flat sheet obtained from the composition of Example 3.1 (DMI) showed the surface covered with very few dead cells visible. Such examples demonstrate that C2C12 cells can attach to, grow on and cover the surface of the flat sheets obtained by the compositions according to the present disclosure.

### Example 6: Hollow Fibres

Set out in Table 3 below is a (non-exhaustive) list of compositions (e.g. a composition used in the method according to the first aspect described herein) from which hollow fibres were obtained. The wt. % amounts of solvent 1 and solvent 2 correspond to the amount present in the solvent of the composition. The wt. % of the polymer corresponds to the amount present in the polymer of the composition. In Example numbers 4.1 to 4.3 and 4.5 to 4.7 and 4.9 to 4.10 the porogen is PEG400, wherein PEG400 is present in the composition in an amount of approximately 10 wt. %. In Example 4.4 and 4.8, the porogen is NaCl, present in the composition in an amount of approximately 7 wt. %.

TABLE 3

| | | Solvent 1 | | Solvent 2 | |
|---|---|---|---|---|---|
| No. | Solvent 1 | Wt. % | Solvent 2 | Wt. % | Temperature |
| | Polystyrene (100 wt. %) | | | | |
| 4.1 | Dimethyl | 100 | N/A | 0 | R.T. |
| | isosorbide | | | | |
| | (DMI) | | | | |
| 4.2 | Acetone | 25 | Dimethyl | 75 | R.T. |
| | | | isosorbide | | |
| | | | (DMI) | | |
| 4.3 | Tamisolve | 100 | N/A | 0 | R.T. |
| 4.4 | Tamisolve | 100 | N/A | 0 | R.T. |
| 4.5 | Acetone | 50 | Tamisolve | 50 | R.T. |
| 4.6 | Polarclean | 100 | N/A | 0 | R.T. |
| | Polysulfone (100 wt. %) | | | | |
| 4.7 | Tamisolve | 100 | N/A | 0 | R.T. |
| 4.8 | Tamisolve | 100 | N/A | 0 | R.T. |
| | Acrylonitrile butadiene styrene (ABS) (100 wt. %) | | | | |
| 4.9 | Tamisolve | 100 | N/A | 0 | R.T. |
| | High impact polystyrene (HIPS) (100 wt. %) | | | | |
| 4.10 | Tamisolve | 100 | N/A | 0 | R.T. |

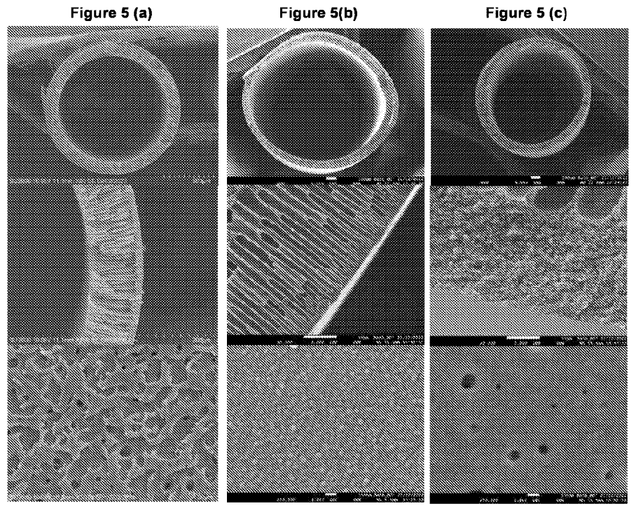

SEM images of (a) micrographs of the cross section of the hollow fibre and (a) surface of the hollow fibre are provided herein for the following Example 4.1 (see FIG. 5 (a)), Example 4.3 (see FIG. 5 (b)) and Example 4.6 (see FIG. 5 (c)).

Example 4.3 was further tested for permeability and exhibited an average permeance of 250 Lm⁻²h⁻¹bar⁻¹. Example 4.3 was also tested for bovine serum albumin (BSA) rejection and had a value of 80%.

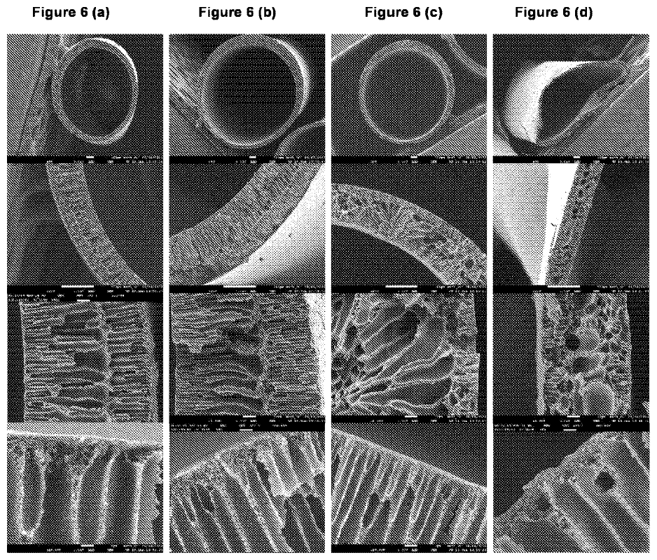

SEM images of micrographs of the cross section of the hollow fibre are provided herein for Example 4.7 (see FIG. 6 (a)), Example 4.3 (see FIG. 6 (b)), Example 4.4 (see FIG. 6 (c)) and Example 4.8 (see FIG. 6 (d)). NaCl containing dopes used PX/PS 18.5%, NaCl 7.4% and Tamisolve (balance). PEG containing dopes used PX/PS 17.5%, PEG10% and Tamisolve (balance).

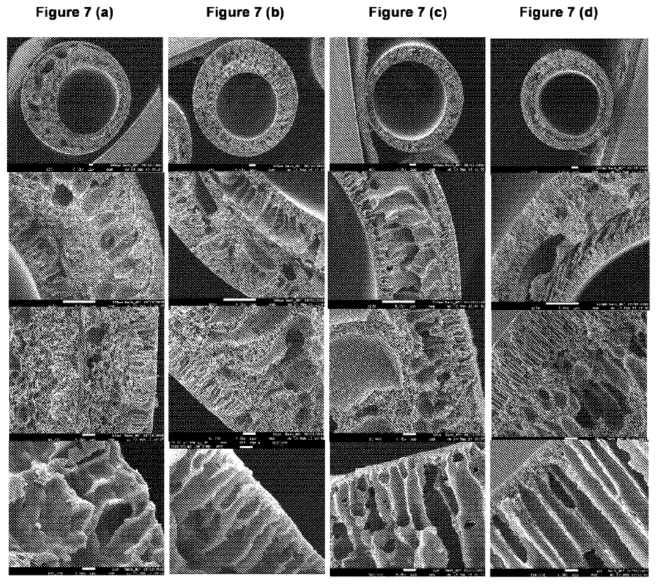

SEM images of micrographs of the cross section of the hollow fibre are provided herein for Example 4.9 (see FIGS. 7(a), 7(b) and 7(c)) and Example 4.10 (see FIG. 7(d)).

### Example 7: Hollow Fibres Used in Bioreactors

A bundle of hollow fibres, obtainable by the method according to the first aspect described herein, were used in a hollow fibre bioreactor (HFB) for the proliferation of C2C12 cells.

HFB reactors were prepared by potting 3 lengths of fibres into glass modules, giving total surface area of approximately 6.2 cm² culture area (outer surface area of fibre). Ethanol was used to sterilise modules before equilibration with basal, then complete growth media. Once equilibration was completed, C2C12 cells were removed from a standard cell culture flask and seeded at 25,000 cell per cm². After attachment, media was recirculated through the lumen of the fibres and allowed to perfuse into the extra-capillary space, supplying the cells with growth medium and dissolved oxygen. Dissolved oxygen was monitored to confirm cells were attached and proliferating, and the reactor continued for 5 days before final cell quantification. Quantification of cells was carried out by excision of fibres and placing into Trypsin EDTA solution before quantification on a Chemometec Nucleocounter NC-200 cell counter.

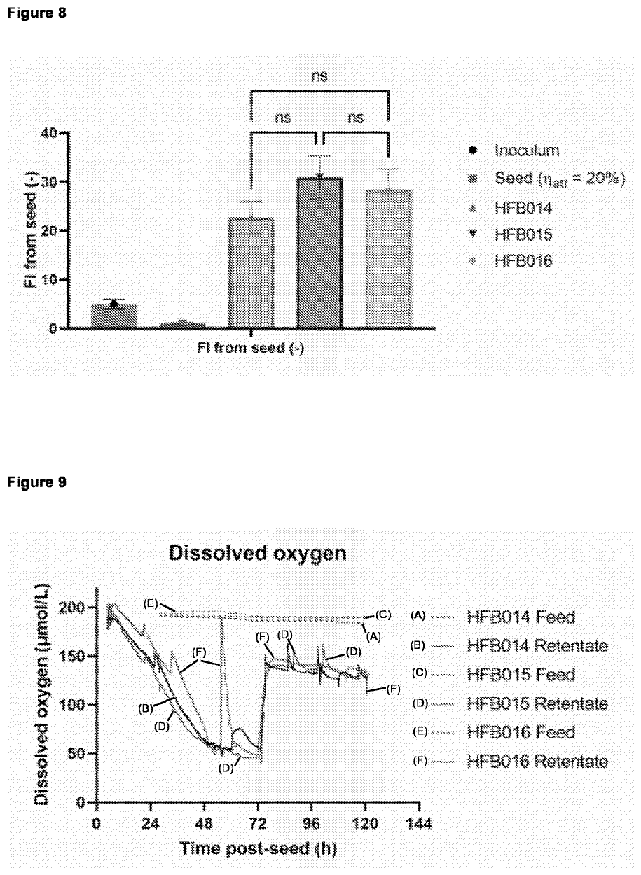

FIG. 8 and FIG. 9 set out the results of reactor runs for a series of hollow fibres from Example 4.3 (corresponding to repeat examples labelled as HFB014, HFB015 and HFB016 in FIG. 8 and FIG. 9). FIG. 8 illustrates a fold increase from seed (20% of the inoculum cell number) after 5 days of proliferation on the fibres within an HFB. FIG. 9 illustrates a graph of the dissolved oxygen profiles which shows the consumption of oxygen concentration in the outlet line as a result of cell growth occurring.

The exemplary hollow fibres demonstrate promising use for the proliferation of C2C12 cells. The methods for manufacturing a hollow fibre according to a first aspect herein can be used for promising tissue engineering applications, including proliferation and/or differentiation of the cells.

### Example 8: Vapour-Induced Phase Separation

Table 4 below shows use of VIPS in the production of hollow fibres. Tested composition for fibres was as follows: Polysulfone as polymer; PVP 10 as porogen and Tamisolve as solvent.

TABLE 4

| | | | Air gap (spinneret | Permeability of | Surface |
|---|---|---|---|---|---|
| | | | to coagulation | membrane | micropore |
| | Formulation | Bore liquid | bath) (cm) | (LMH/bar) | presence |
| (a) | 17.5% | 25% | 4 | Below detection | None |
| | Polysulfone | Tamisolve | | limit | |
| | 10% PVP | 75% water | | | |
| | 82.5% | | | | |
| | Tamisolve | | | | |
| (b) | 17.5% | 25% | 65 | 40.3 | Yes |
| | Polysulfone | Tamisolve | | | |
| | 10% PVP | 75% water | | | |
| | 82.5% | | | | |
| | Tamisolve | | | | |

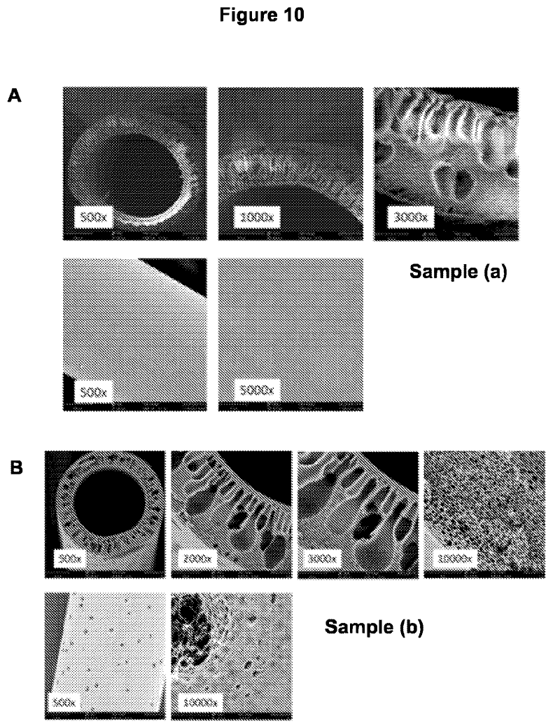

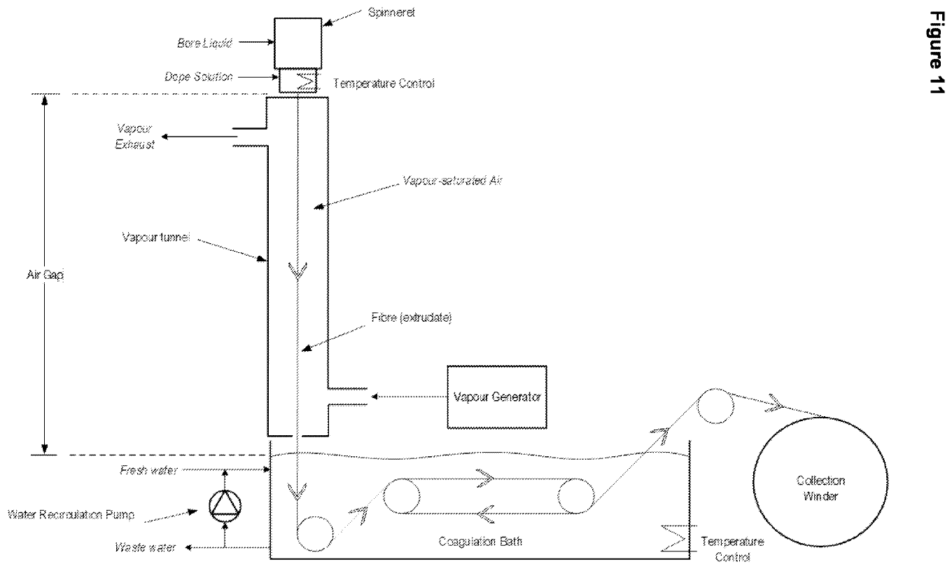

It can be seen from FIG. 10 that sample (b) had micropores on the surface.

It will be appreciated by persons skilled in the art that the above detailed examples have been described by way of example only and not in any limitative sense, and that various alterations and modifications are possible without departing from the scope of the invention as defined by the appended claims. Various modifications to the detailed examples described above are possible.

Through the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract or drawings), or to any novel one, or any novel combination, of the steps of any method or process so disclosed.

It will be appreciated by persons skilled in the art that the above embodiment(s) have been described by way of example only and not in any limitative sense, and that various alterations and modifications are possible without departing from the scope of the invention as defined by the appended claims. Various modifications to the detailed designs as described above are possible.

### CLAUSES

1. A method for manufacturing a hollow fibre for tissue engineering, comprising a step of extruding a composition for forming a hollow fibre, wherein the composition comprises:
   a polymer;
   a porogen; and
   a non-toxic solvent.
2. The method according to clause 1, wherein the non-toxic solvent is a non-cytotoxic solvent and/or a non-CMR solvent.
3. The method according to any preceding clause, wherein the composition is extruded with a nonsolvent agent, optionally wherein the nonsolvent agent comprises water.
4. The method according to clause 3, wherein the composition is extruded in a coagulating bath, wherein the coagulating bath comprises the nonsolvent agent.
5. The method according to any preceding clause, wherein the non-toxic solvent is aprotic, preferably polar aprotic.
6. The method according to any preceding clause, wherein the non-toxic solvent comprises an oxygen-containing functional group.
7. The method according to any preceding clause, wherein the non-toxic solvent is selected from the group consisting of acetone; dimethyl sulfoxide (DMSO); dimethyl isosorbide (DMI); d-limonene; cyrene (dihydrolevoglucosenone); methyl oleate; triethyl phosphate (TEP); γ-butyrolactone; 1,2-butylene carbonate; methyl ethyl ketone (MEK); palm oil; cyclopentanone; dioctyl adipate; isophorone; acetyl triethyl citrate (ATEC); oleic acid; ethyl lactate; methyl lactate; acetyl tributyl citrate; triethylcitrate; triethylene glycol diacetate; triethyleneglycol diacetate acetone; γ-valerolactone; ethyl acetate; propan-2-ol (IPA); ethanol; methyl 5-(dimethylamino)-2-methyl-5-oxopentanoate (e.g. Rhodiasolv™ Polarclean (CAS-No. 174627-68-9)); N-butyl-2-pyrrolidinone (e.g. TamiSolve™ NxG); and a mixture of two or more thereof.
8. The method according to any preceding clause, wherein the non-toxic solvent is selected from the group consisting of acetone; dimethyl sulfoxide (DMSO); dimethyl isosorbide (DMI); d-limonene; methyl 5-(dimethylamino)-2-methyl-5-oxopentanoate (e.g. Rhodiasolv™ Polarclean (CAS-No. 174627-68-9)); N-butyl-2-pyrrolidinone (e.g. TamiSolve™ NxG); and a mixture of two or more thereof.
9. The method according to any preceding clause, wherein the non-toxic solvent comprises methyl 5-(dimethylamino)-2-methyl-5-oxopentanoate.
10. The method according to any preceding clause, wherein the non-toxic solvent comprises N-alkyl-2-pyrrolidone, wherein the alkyl group is selected from C2-C12, optionally wherein the non-toxic solvent comprises N-butyl-2-pyrrolidinone.
11. The method according to any preceding clause, wherein the hollow fibre comprises no more than 10 wt. % of residual solvent, optionally less than 1 wt. % of residual solvent.
12. The method according to any preceding clause, wherein the polymer is selected from the group consisting of polystyrene, polycarbonate, polypropylene, polyvinylidene fluoride, polycaprolactone, polysulfone, cellulose acetate, cellulose triacetate, polyethersulfone, polyphenylenesulfon, polymethyl methacrylate, acrylonitrile butadiene styrene or mixtures thereof.
13. The method according to any preceding clause, wherein the polymer comprises polystyrene, polysulfone, high impact polystyrene (HIPs), or mixtures thereof
14. The method according to any preceding clause, wherein the polymer and the porogen are present in a ratio of about 1:0.01 to 10, preferably 1:0.5 to 5, more preferably 1:0.1 to 3, even more preferably 1:0.1 to 1.5.
15. The method according to any preceding clause, wherein the porogen is present in an amount of less than 60 wt. %, preferably less than 30 wt. %, more preferably less than 15 wt. %.
16. The method according to any preceding clause, wherein the porogen comprises salt particles, optionally wherein the salt particles comprise sodium chloride (NaCl).
17. The method according to any preceding clause, wherein the porogen comprises polyethylene glycol (PEG).
18. The method according to any preceding clause, wherein the polyethylene glycol (PEG) has a number average molecular weight of about 300 g/mol to about 20,000 g/mol, optionally wherein the porogen comprises PEG 400.
19. The method according to any preceding clause, wherein the hollow fibre has an average pore diameter from about 0.001 μm to about 100 μm, optionally having an average pore diameter from about 0.001 μm to about 5 μm.
20. A composition for forming a hollow fibre for tissue engineering, comprising
   a polymer;
   a porogen; and
   a non-toxic solvent.
21. The composition according to clause 20, wherein the composition is used in the method according to clauses 1-19.
22. A hollow fibre suitable for use in tissue engineering scaffolds, obtainable from the method according to clauses 1-19 or obtainable from the composition according to clauses 20-21.
23. Use of a hollow fibre according to clause 22 in tissue engineering scaffolds.
24. Use of a hollow fibre according to clause 22 in an application of cell proliferation, cell differentiation and/or culturing or harvesting of cells.
25. Use of a hollow fibre according to clause 22 in a method of culturing cells.

1. A method for manufacturing a hollow fibre for tissue engineering, comprising a step of extruding a composition for forming a hollow fibre, wherein the composition comprises:
a polymer;
a porogen; and
a non-toxic solvent;
wherein the non-toxic solvent comprises one or more of dimethyl sulfoxide (DMSO); dimethyl isosorbide (DMI); d-limonene; cyrene (dihydrolevoglucosenone); γ-butyrolactone; isophorone; ethyl lactate; methyl lactate; γ-valerolactone; ethyl acetate; propan-2-ol (IPA); ethanol; methyl 5-(dimethylamino)-2-methyl-5-oxopentanoate; cyclopentanone; and/or N-butyl-2-pyrrolidinone.

2. The method according to claim 1, wherein the non-toxic solvent is a non-cytotoxic solvent and/or a non-CMR solvent.

3. The method according to claim 1 or 2, wherein the non-toxic solvent comprises methyl 5-(dimethylamino)-2-methyl-5-oxopentanoate.

4. The method according to any preceding claim, wherein the non-toxic solvent comprises N-butyl-2-pyrrolidinone.

5. The method according to any preceding claim, wherein the polymer comprises one or more of polystyrene, polysulfone, high impact polystyrene (HIPs), polycaprolactone, cellulose acetate, cellulose triacetate, polyethersulfone, acrylonitrile butadiene styrene, sulfonated polymers, polymethyl methacrylate (PMMA), poly(lactic acid) (PLA), poly(lactic-co-glycolic acid) (PLGA), and/or polyhydroxyalkanoate (PHA).

6. The method according to any preceding claim, wherein the polymer comprises polystyrene, polysulfone, high impact polystyrene (HIPs), or mixtures thereof.

7. The method according to any preceding claim, wherein the composition is extruded with a nonsolvent agent, optionally wherein the nonsolvent agent comprises water.

8. The method according to claim 7, wherein the composition is extruded in a coagulating bath, wherein the coagulating bath comprises the nonsolvent agent.

9. The method according to claim 8, wherein prior to the extruding in a coagulating bath the method comprises exposing the composition to a vapor, the vapour comprising a non-solvent.

10. The method according to any preceding claim, wherein the non-toxic solvent is aprotic, preferably polar aprotic.

11. The method according to any preceding claim, wherein the non-toxic solvent comprises an oxygen-containing functional group.

12. The method according to any preceding claim, wherein the hollow fibre comprises no more than 10 wt. % of residual solvent, optionally less than 1 wt. % of residual solvent.

13. The method according to any preceding claim, wherein the polymer and the porogen are present in a ratio of about 1:0.01 to 10, preferably 1:0.5 to 5, more preferably 1:0.1 to 3, even more preferably 1:0.1 to 1.5.

14. The method according to any preceding claim, wherein the porogen is present in the composition at an amount of less than 60 wt. %, preferably less than 30 wt. %, more preferably less than 15 wt. %.

15. The method according to any preceding claim, wherein the porogen comprises salt particles, optionally wherein the salt particles comprise sodium chloride (NaCl).

16. The method according to any preceding claim, wherein the porogen comprises polyethylene glycol (PEG).

17. The method according to any preceding claim, wherein the polyethylene glycol (PEG) has a number average molecular weight of about 300 g/mol to about 20,000 g/mol, optionally wherein the porogen comprises PEG 400.

18. The according to any preceding claim, wherein the porogen comprises polyvinylpyrrolidone (PVP).

19. The method according to any preceding claim, wherein the hollow fibre has an average pore diameter from about 0.1 μm to about 5 μm.

20. The method according to any preceding claim, wherein the method further comprises subjecting the hollow fibres to a post-synthesis surface modification selected from chemical surface modification, coating and/or plasma modification.

21. A composition for forming a hollow fibre for tissue engineering, comprising a polymer;
a porogen; and
a non-toxic solvent;
wherein the non-toxic solvent comprises one or more of dimethyl sulfoxide (DMSO); dimethyl isosorbide (DMI); d-limonene; cyrene (dihydrolevoglucosenone); γ-butyrolactone; isophorone; ethyl lactate; methyl lactate; γ-valerolactone; ethyl acetate; propan-2-ol (IPA); ethanol; methyl 5-(dimethylamino)-2-methyl-5-oxopentanoate; cyclopentanone; N-butyl-2-pyrrolidinone.

22. The composition according to claim 21, wherein the composition is used in the method according to claims 1-20.

23. A hollow fibre suitable for use in tissue engineering scaffolds, obtainable from the method according to claims 1-20 or obtainable from the composition according to claims 21-22.

24. Use of a hollow fibre according to claim 23 in tissue engineering scaffolds.

25. Use of a hollow fibre according to claim 23 in an application of cell proliferation, cell differentiation and/or culturing or harvesting of cells.

26. Use of a hollow fibre according to claim 23 in a method of culturing cells.
